(19) United States
(12) Patent Application Publication
(10) Pub. No.: US 2013/0023693 A1
(43) Pub. Date: Jan. 24, 2013

(54) COMPOUND HAVING ALICYCLIC STRUCTURE, (METH)ACRYLIC ACID ESTER, AND PROCESS FOR PRODUCTION OF THE (METH)ACRYLIC ACID ESTER

(71) Applicant: IDEMITSU KOSAN CO., LTD., Tokyo (JP)

(72) Inventors: Shinji Tanaka, Chiba (JP); Kazuya Fukushima, Chiba (JP); Katsuki Ito, Chiba (JP); Naoya Kawano, Chiba (JP); Hideki Yamane, Chiba (JP)

(73) Assignee: IDEMITSU KOSAN CO., LTD., Tokyo (JP)

(21) Appl. No.: 13/631,090

(22) Filed: Sep. 28, 2012

Related U.S. Application Data
(62) Division of application No. 12/918,667, filed on Aug. 23, 2010.

(30) Foreign Application Priority Data
Feb. 22, 2008 (JP) 2008-041139
May 14, 2008 (JP) 2008-127294

(51) Int. Cl. C07C 69/54

(52) U.S. Cl. 560/220

(57) ABSTRACT

Provided are an alicyclic structure-containing compound, a (meth)acrylate, and a method for producing the ester. The compound and the ester are useful as a monomer and the like for a photoresist used in semiconductor manufacturing and excellent in solubility, compatibility, defect reduction, roughness improvement, and the like, realized by using an alicyclic structure-containing compound containing a linking group having an ester bond and/or a linking group having an ether bond, a (meth)acrylate derived from the alicyclic structure-containing compound, and a method for producing the ester.

### TECHNICAL FIELD

The present invention relates to a novel alicyclic structure-containing compound, a (meth)acrylate, and a method for producing the ester. More specifically, the present invention relates to an alicyclic structure-containing compound, a (meth)acrylate, and a method for producing the same, the compound and the ester having an alicyclic structure and being excellent in solubility, compatibility, defect reduction, roughness improvement, and the like.

### BACKGROUND ART

In recent years, with progress in miniaturization of semiconductor devices, even finer patterning is required in a photolithography step of semiconductor manufacturing. Thus, many methods are under study how to form fine patterns using photoresist materials corresponding to short-wavelength irradiation light such as KrF, ArF, F₂ excimer laser light or the like. Accordingly, desired is appearance of a novel photoresist material that can correspond to short-wavelength irradiation light such as the excimer laser light or the like.

As a photoresist material, there have heretofore been developed many based on phenol resins. However, these materials absorb light strongly because they contain aromatic rings and, therefore, it is not possible with these materials to achieve patterning accuracy that can meet requirements of fine patterning.

For this reason, as a photosensitive resist used in semiconductor manufacturing by using an ArF excimer laser, there has been proposed a polymer obtained by copolymerization of a polymerizable compound having an alicyclic structure such as 2-methyl-2-adamantyl methacrylate (see, for example, Patent Document 1).

With further progress in microfabrication technology, realization of a line-width of 32 nm or less is currently under study. However, with conventional technology alone, it has not been possible to achieve various performance requirements such as exposure sensitivity, resolution, pattern shapes, exposure depth, surface roughness, and the like. Specifically, problems related to smoothness such as surface roughness termed LER and LWR, and undulation have become apparent. Further, in a recent method by immersion exposure, there are occasionally found cases of poor development such as defects in the resist pattern, which are attributable to the immersion medium. Solutions to these problems are urgently desired.

Under these circumstances, attempts have been made to improve LER using a resist material which uses a glycolate containing many carbonyl groups (see Patent Document 2) or to improve solubility in a resist solvent by attaching a long alkylene chain to the stiff (meth)acrylic acid main chain (see Patent Document 3). However, with these techniques alone, it is difficult to achieve the aforementioned performance requirements and, thus, further improvement of the resist is required

### [Patent Document 1]

Japanese Patent Laid-Open Publication No. H4-39665

### [Patent Document 2]

Japanese Patent Laid-Open Publication No. 2005-331918

### [Patent Document 3]

Japanese Patent No. 3952946

### DISCLOSURE OF THE INVENTION

### Problems to be Solved by the Invention

Under the above-described circumstances, it is the object of the present invention to provide an alicyclic structure-containing compound, a (meth)acrylate, and a method for producing the ester, the compound and the ester being useful as a monomer and the like for a photoresist used in semiconductor manufacturing and being excellent in solubility, compatibility, defect reduction, roughness improvement, and the like.

### Means for Solving the Problems

The present inventors conducted diligent research and, as a result, have found that a (meth)acrylate derived from an alicyclic structure-containing compound, having many carbonyl groups and ester bonds and thus having oxygen-containing groups actively incorporated, can, when copolymerized, exhibit better compatibility and higher solubility in a resist solvent; also that solubility of the copolymer in an alkaline developer is improved after exposure, thus leading to reduction of defects; and further that, by elongating the main chain of the (meth)acrylic acid and the terminal group, it becomes possible to regulate polymerizability without being affected by the terminal group and to narrow molecular weight distribution, thus making it possible to improve roughness. The present invention was completed based on these findings.

According to the present invention, there are provided:

1. an alicyclic structure-containing compound represented by the following general formula (I):

R¹-L-X (I)

wherein R¹ represents an alicyclic structure-containing group having 5 to 20 carbon atoms, represented by the following general formula (i); L represents a linking group represented by the following general formula (ii); and X represents a halogen atom or a hydroxyl group;

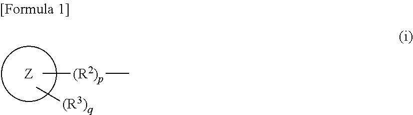

wherein Z represents an alicyclic structure having 5 to 20 carbon atoms optionally containing a heteroatom; R² represents a substituted or unsubstituted bivalent hydrocarbon group having 1 to 5 carbon atoms optionally containing a heteroatom; R³ represents a substituted or unsubstituted alkyl group optionally containing a heteroatom, a halogen atom, a hydroxyl group, a cyano group, a carboxyl group, an oxo group, or an amino group; p and q each independently represent an integer equal to or larger than 0; plural R²'s may be the same or different and plural R³'s may be the same or different;

-{(L^{a})ₗ,(L^{b})ₘ,(L_{c})ₙ}- (ii)

wherein L^{a} represents a linking group represented by the following formula (a); L^{b} represents a linking group represented by the following formula (b); L^{c} represents a linking group represented by the following formula (c); and L^{a}, L^{b}, and L^{c} may be bound in any order; and l, m, and n represent each independently an integer equal to or larger than 0 and satisfy l+m+n≧2;

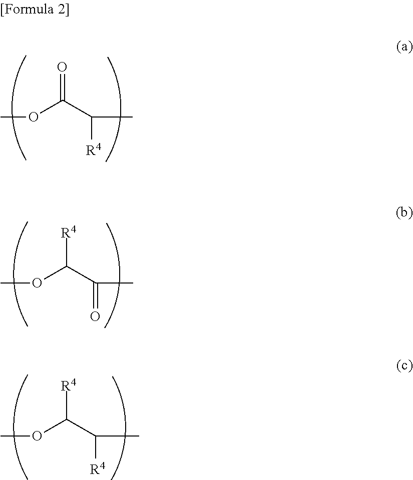

wherein R⁴'s each independently represent a hydrogen atom or a methyl group;
2. the alicyclic structure-containing compound according to the above item 1, represented by any one of the following general formulae (1) to (9):

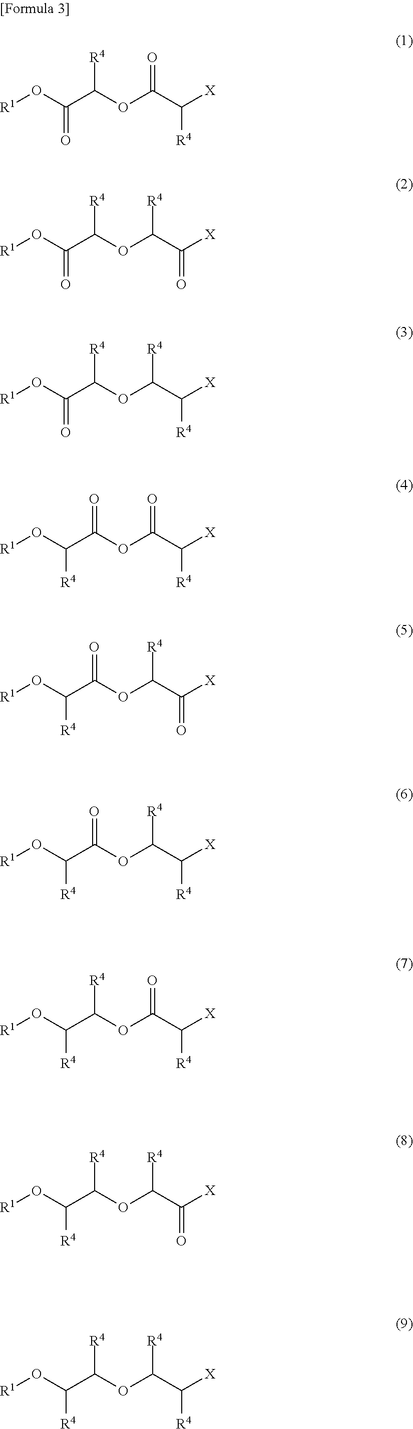

wherein R¹ represents an alicyclic structure-containing group having 5 to 20 carbon atoms, represented by the above general formula (i); R⁴'s represent each independently a hydrogen atom or a methyl group; and X represents a halogen atom or a hydroxyl group;
3. a (meth)acrylate represented by the following general formula (II):

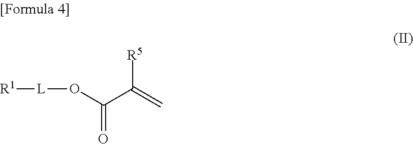

wherein R¹ represents an alicyclic structure-containing group having 5 to 20 carbon atoms, represented by the following general formula (i); R⁵ represents a hydrogen atom, a methyl group, a fluorine atom, or a trifluoromethyl group; and L represents a linking group represented by the following general formula (ii);

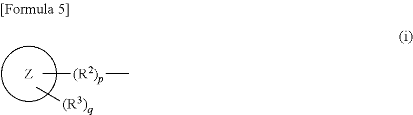

wherein Z represents an alicyclic structure having 5 to 20 carbon atoms optionally containing a heteroatom; R² represents a substituted or unsubstituted bivalent hydrocarbon group having 1 to 5 carbon atoms optionally containing a heteroatom; R³ represents a substituted or unsubstituted alkyl group optionally containing a heteroatom, a halogen atom, a hydroxyl group, a cyano group, a carboxyl group, an oxo group, or an amino group; p and q each independently represent an integer equal to or larger than 0; plural R²'s may be the same or different; and plural R³'s may be the same or different;

-{(L^{a})ₗ,(L^{b})ₘ,(L_{c})ₙ}- (ii)

wherein 12 represents a linking group represented by the following formula (a); L^{b} represents a linking group represented by the following formula (b); L^{c} represents a linking group represented by the following formula (c); and L^{a}, L^{b}, and L^{c} may be bound in any order, and l, m, and n represent each independently an integer equal to or larger than 0 and satisfy l+m+n≧2;

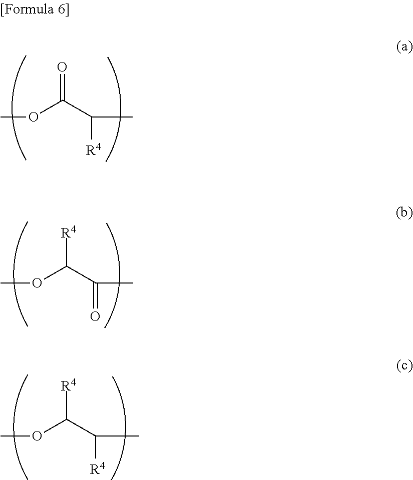

wherein R⁴'s each independently represent a hydrogen atom or a methyl group;
4. the (meth)acrylate according to the above item 3, represented by any one of the following formulae (10) to (18):

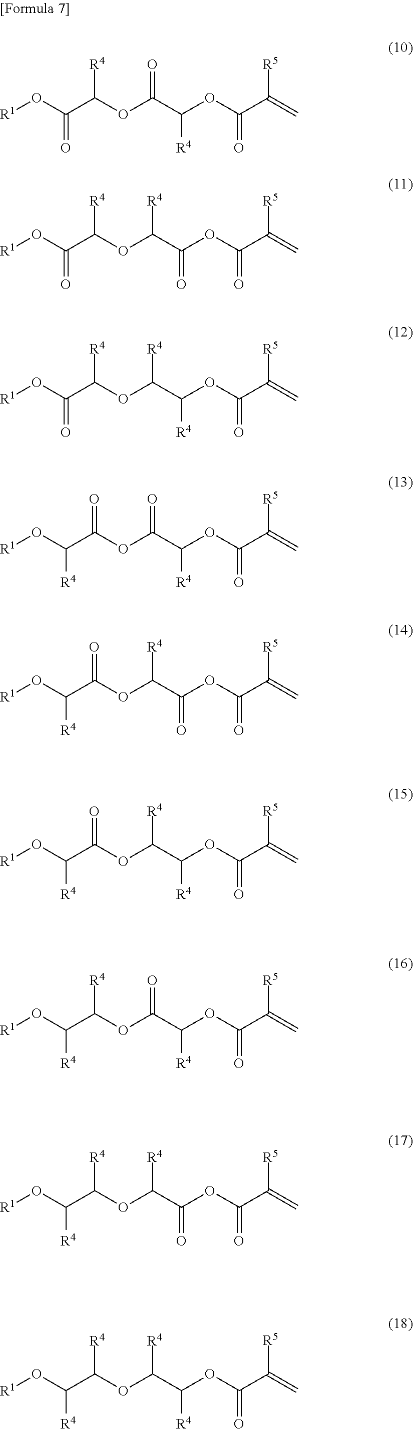

wherein R¹ represents an alicyclic structure-containing group having 5 to 20 carbon atoms, represented by the above general formula (i); R⁴'s each independently represent a hydrogen atom or a methyl group; and R⁵ represents a hydrogen atom, a methyl group, a fluorine atom, or a trifluoromethyl group;
5. the (meth)acrylate according to the above item 3 or 4 wherein the Z is an adamantyl ring;
6. the (meth)acrylate according to the above item 5 wherein, in the formula (II), l+n=2 and m=0;
7. the (meth)acrylate according to the above item 6 wherein the L represents a linking group represented by the following general formula (iii):

-L^{a}-L^{a}- (iii)

wherein L^{a} represents a linking group represented by the above formula (a);
8. a method for producing a (meth)acrylate wherein the (meth)acrylate according to any one of the above items 3 to 7 is obtained by esterification of the alicyclic structure-containing compound according to the above item 1 or 2 and one or more selected from (meth)acrylic acid, a (meth)acrylic acid halide, and a (meth)acrylic anhydride; and
9. a method for producing a (meth)acrylate wherein the (meth)acrylate according to any one of the above items 3 to 7 is obtained by transesterification of an alicyclic structure-containing (meth)acrylic acid and a dilactide by a ring-opening reaction.

The (meth)acrylate derived from the alicyclic structure-containing compound of the present invention is excellent in solubility, compatibility, defect reduction, roughness improvement, and the like.

### BEST MODE FOR CARRYING OUT THE INVENTION

The alicyclic structure-containing compound of the present invention is represented by the following general formula (I):

R¹-L-X (I)

wherein R¹ represents an alicyclic structure-containing group having 5 to 20 carbon atoms, represented by the following general formula (i); L represents a linking group represented by the following general formula (ii); and X represents a halogen atom or a hydroxyl group:

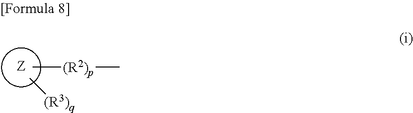

wherein Z represents an alicyclic structure having 5 to 20 carbon atoms, preferably 7 to 12 carbon atoms optionally containing a heteroatom, more preferably an adaniantyl ring; R² represents a substituted or unsubstituted bivalent hydrocarbon group having 1 to 5 carbon atoms optionally containing a heteroatom, preferably a bivalent hydrocarbon group having 1 to 2 carbon atoms; R³ represents a substituted or unsubstituted alkyl group optionally containing a heteroatom, a halogen atom, a hydroxyl group, a cyano group, a carboxyl group, an oxo group, or an amino group, preferably a halogen atom, a hydroxyl group, or an oxo group; p represents an integer equal to or larger than 0, preferably 0 to 5, more preferably 0 to 2; q represents an integer equal to or larger than 0, preferably 0 to 20, more preferably 0 to 15; plural R²'s may be the same or different; and plural R³'s may be the same or different;

-{(L^{a})ₗ,(L^{b})ₘ,(L_{c})ₙ}- (ii)

wherein L^{a} represents a linking group represented by the following formula (a); L^{b} represents a linking group represented by the following formula (b); L^{c} represents a linking group represented by the following formula (c); L^{a}, L^{b}, and L^{c} may be bound in any order; and l, m, and n represent each independently an integer equal to or larger than 0 and satisfy l+m+n≧2;

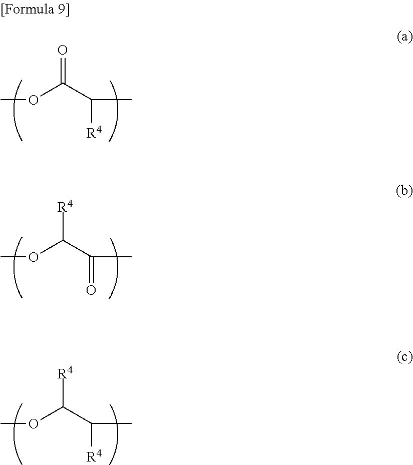

wherein R⁴'s each independently represent a hydrogen atom or a methyl group.

The halogen atom in the above formula (I) includes a fluorine atom, a chlorine atom, a bromine atom, and an iodine atom.

The alicyclic structure in the above formula (I), having 5 to 20 carbon atoms optionally containing a heteroatom, includes, for example, monocyclic or polycyclic structures such as a cyclopeptyl ring, a cyclohexyl ring, a cycloheptyl ring, a cyclooctyl ring, a cyclononyl ring, a cyclodecanyl ring, a decalyl ring (perhydronaphthalene ring), a norbornyl ring, a bornyl ring, an isobornyl ring, an adarnantyl ring, a tricyclo[5.2, 1.0^{2,6}]decane ring, a tetracyclo[4.4.0.1^{2,5}.1^{7,10}]dodecane ring, and the like; monocyclic or polycyclic lactones such as a γ-butyrolactyl ring, 4-oxa-tricyclo[4.2.1.0^{3,7}]nonan-5-one, 4,8-dioxa-trieyclo[4.2.1.0^{3,7}]nonan-5-one, 4-oxa-tricyclo[4.3.1.1^{3,8}]undecan-5-one, and the like; and perfluoro derivatives of these.

Specific examples of the substituted or unsubstituted bivalent hydrocarbon group in the above formula (i), having 1 to 5 carbon atoms optionally containing a heteroatom, include linear or branched alkylene groups such as a methylene group, an ethylene group, a trimethylene group, and the like; and perfluoro derivatives of these.

Specific examples of the substituted or unsubstituted alkyl group in the above formula (I) optionally containing a heteroatom, include linear or branched alkyl groups such as a methyl group, an ethyl group, a n-propyl group, an isopropyl group, a n-butyl group, a sec-butyl group, a tert-butyl group, a n-pentyl group, an isopentyl group, a hexyl group, a heptyl group, an octyl group, a nonyl group, a decanyl group, and the like; and perfluoro derivatives of these.

Specific examples of the above heteroatom, which may be contained in the alicyclic structure having 5 to 20 carbon atoms optionally containing a heteroatom, the substituted or unsubstituted bivalent hydrocarbon group having 1 to 5 carbon atoms optionally containing a heteroatom, or a substituted or unsubstituted alkyl group optionally containing a heteroatom therein, include a nitrogen atom, a sulfur atom, an oxygen atom, and the like.

L in the above general formula (I) represents a bivalent linking group represented by the above general formula (ii) and is composed of the above linking groups L^{a}, L^{b}, and L^{c}. These linking groups may be bound in any order to constitute the linking group L. When the linking group L contains at least a plurality of any one of L^{a}, L^{b}, and L^{c}, the respective linking groups L^{a}'s, L^{b}'s, and L^{c}'s may be the same with or different from each other. In addition, the linking groups of the same kind need not be bound next to each other and, specifically, the binding order may be like L^{a}-L^{b}-L^{c}.

In the above general formula (ii), l, m, and n satisfy l+m+n≧2, preferably l+m+n=2, more preferably l+n=2 and m=0.

The above L is most preferably a linking group represented the following general formula (iii):

-L^{a}-L^{a}- (iii)

wherein L^{a} represents a linking group represented by the above formula (a).

In addition, the alicyclic structure-containing compound of the present invention is preferably one represented by any one of the following general formulae (1) to (9):

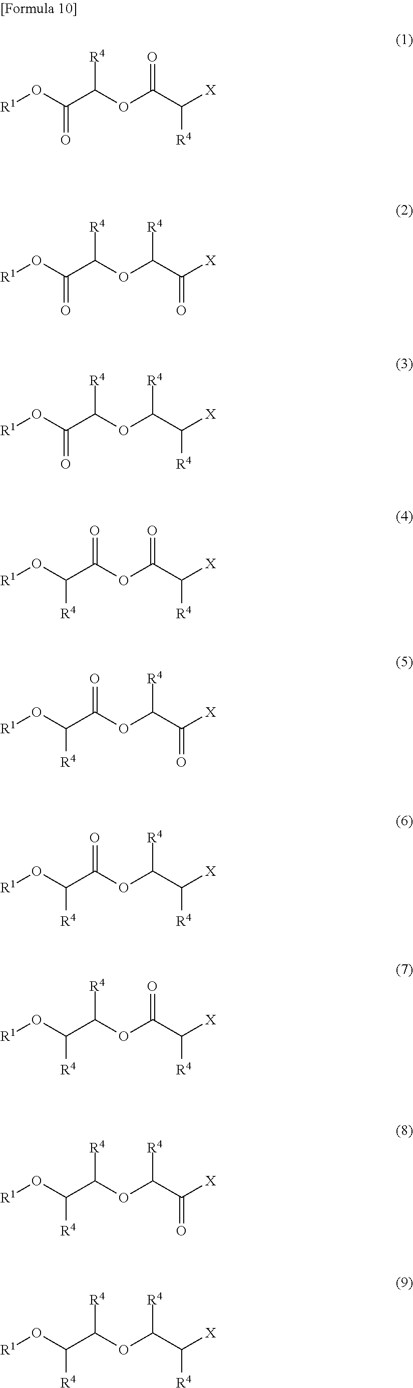

wherein R¹ represents an alicyclic structure-containing group having 5 to 20 carbon atoms, represented by the above general formula (i); R⁴'s each independently represent a hydrogen atom or a methyl group; and X represents a halogen atom or a hydroxyl group.

The halogen atoms in the above formulae (1) to (9) include a fluorine atom, a chlorine atom, a bromine atom, and an iodine atom.

Specific examples of the alicyclic structure-containing compound of the present invention, represented by the above formulae (1) to (9), include: 2-(2-(cyclopentyloxy)-2-oxoethoxy)-2-oxoethanol, 2-(2-(cyclohexyloxy)-2-oxoethoxy)-2-oxoethanol, 2-(2-(cycloheptyloxy)-2-oxoethoxy)-2-oxoethanol, 2-(2-(cyclooctyloxy)-2-oxoethoxy)-2-oxoethanol, 2-(2-(cyclononyloxy)-2-oxoethoxy)-2-oxoethanol, 2-(2-(cyclodecanyloxy)-2-oxoethoxy)-2-oxoethanol, 2-(2-(cyclodecalyloxy)-2-oxoethoxy)-2-oxoethanol, 2-(2-(norbornyloxy)-2-oxoethoxy)-2-oxoethanol, 2-(2-(bornyloxy)-2-oxoethoxy)-2-oxoethanol, 2-(2-(isobornyloxy)-2-oxoethoxy)-2-oxoethanol, 2-(2-(1-adamantyloxy)-2-oxoethoxy)-2-oxoethanol,

2-(2-(3-tricyclo[5.2.1.0^{2,6}]decanyloxy)-2-oxoethoxy)-2-oxoethanol, 2-(2-(3-tetracyclo[4.4, 0.1^{2,5}.1^{7,10}]dodecanyloxy)-2-oxoethoxy)-2-oxoethanol, 2-(2-(1-γ-butyrolactyloxy)-2-oxoethoxy)-2-oxoethanol, 2-(2-(5-(2,6-norbornanecarbolactyl)oxy)-2-oxoethoxy)-2-oxoethanol, 2-(2-(5-(7-oxa-2,6-norbornanecarbolactyl)oxy)-2-oxoethoxy)-2-oxoethanol, 2-(2-(8-(4-oxa-tricyclo[4.3.1.1^{3,8}]undecan-5-one)oxy)-2-oxoethoxy)-2-oxoethanol, 2-(2-(1-adamantylmethoxy)-2-oxoethoxy)-2-oxoethanol, 2-(2-(2-(1-adamantylethoxy)-2-oxoethoxy)-2-oxoethanol, 2-(2-(2-adamantyloxy)-2-oxoethoxy)-2-oxoethanol, 2-(2-(2-methyl-2-adamantyloxy)-2-oxoethoxy)-2-oxoethanol, 2-(2-(2-ethyl-2-adamantyloxy)-2-oxoethoxy)-2-oxoethanol, 2-(2-(2-isopropyl-2-adamantyloxy)-2-oxoethoxy)-2-oxoethanol, 2-(2-(3-hydroxy-1-adamantyloxy)-2-oxoethoxy)-2-oxoethanol, 2-(2-(3,5-dihydroxy-1-adamantyloxy)-2-oxoethoxy)-2-oxoethanol, 2-(2-(3-hydroxymethyl-1-adamantylmethoxy)-2-oxoethoxy)-2-oxoethanol, 2-(2-(3-carboxyl-1-adamantylmethoxy)-2-oxoethoxy)-2-oxo ethanol, 2-(2-(2-cyanomethyl-2-adamantyloxy)-2-oxoethoxy)-2-oxoethanol, 2-(2-(4-oxo-2-adamantyloxy)-2-oxoethoxy)-2-oxoethanol, 2-(2-(1-adamatyl)dimethylmethoxy-2-oxo ethoxy)-2-oxo ethanol, 2-(2-(5-oxo-2-adamantyloxy)-2-oxoethoxy)-2-oxoethanol, 2-(2-(perfluorocyclopentyloxy)-2-oxoethoxy)-2-oxoethanol,

2-(2-(perfluorocyclohexyloxy)-2-oxoethoxy)-2-oxoethanol, 2-(2-(perfluoro-1-adamantyloxy)-2-oxo ethoxy)-2-oxoethanol, 2-(2-(3-hydroxy-perfluoro-1-adamantyloxy)-2-oxoethoxy)-2-oxoethanol, 2-(2-(perfluoro-1-adamantylmethoxy)-2-oxoethoxy)-2-oxoethanol, 2-(2-(3-hydroxy-perfluoro-1-adamantylmethoxy)-2-oxoethoxy)-2-oxoethanol, 2-(1-methyl-2-(2-methyl-2-adamantyloxy)-2-oxo ethoxy)-1-methyl-2-oxoethanol, 2-(1-methyl-2-(2-ethyl-2-adamantyloxy)-2-oxoethoxy)-1-methyl-2-oxo ethanol, 2-(1-methyl-2-(2-isopropyl-2-adamantyloxy)-2-oxoethoxy)-1-methyl-2-oxoethanol, 2-(2-(2-methyl-2-adamantyloxy)-2-oxoethoxy)-1-oxoethanol, 2-(2-(2-methyl-2-adamantyloxy)-2-oxoethoxy)ethanol, 2-(2-(2-methyl-2-adamantyloxy)-1-oxoethoxy)-2-oxoethanol, 2-(2-(2-methyl-2-adamantyloxy)-1-oxoethoxy)-1-oxoethanol, 2-(2-(2-methyl-2-adamantyloxy)-1-oxoethoxy)ethanol, 2-(2-(2-methyl-2-adamantyloxy)ethoxy)-2-oxoethanol, 2-(2-(2-methyl-2-adamantyloxy)ethoxy)-1-oxoethanol, 2-(2-(2-methyl-2-adamantyloxy)ethoxy)ethanol,

2-(2-(perfluoro-1-adamantyloxy)ethoxy)-2-oxoethanol, 2-(2-(3-hydroxy-perfluoro-1-adamantyloxy)ethoxy)-2-oxoethanol, 2-(2-(1-adamantyl)dimethylmethoxyethoxy)-2-oxoethanol, 2-(2-(5-(2,6-norbornanecarbolactyl)oxy)ethoxy)-2-oxoethanol, 2-(2-(5-(7-oxa-2,6-norbornanecarbolactyl)oxy)ethoxy)-2-oxoethanol, 2-(2-(cyclopentyloxy)-2-oxoethoxy)-2-oxoethyl bromide, 2-(2-(cyclohexyloxy)-2-oxoethoxy)-2-oxoethyl bromide, 2-(2-(cycloheptyloxy)-2-oxoethoxy)-2-oxoethyl bromide, 2-(2-(cyclooctyloxy)-2-oxoethoxy)-2-oxoethyl bromide, 2-(2-(cyclononyloxy)-2-oxoethoxy)-2-oxoethyl bromide, 2-(2-(cyclodecanyloxy)-2-oxoethoxy)-2-oxoethyl bromide, 2-(2-(cyclodecalyloxy)-2-oxoethoxy)-2-oxoethyl bromide, 2-(2-(norbornyloxy)-2-oxoethoxy)-2-oxoethyl bromide, 2-(2-(bornyloxy)-2-oxoethoxy)-2-oxoethyl bromide, 2-(2-(isobornyloxy)-2-oxoethoxy)-2-oxoethyl bromide, 2-(2-(1-adamantyloxy)-2-oxoethoxy)-2-oxoethyl bromide, 2-(2-(3-tricyclo[5.2.1.0^{2,6}]decanyloxy)-2-oxoethoxy)-2-oxoethyl bromide, 2-(2-(3-tricylo[4.4.0.1^{2,5}1^{7,10}]dodecanyloxy)-2-oxoethoxy)-2-oxoethyl bromide,

2-(2-(1-γ-butyrolaotyloxy)-2-oxoethoxy)-2-oxoethyl bromide, 2-(2-(5-(2,6-norbornanecarbolactyl)oxy)-2-oxoethoxy)-2-oxoethyl bromide, 2-(2-(5-(7-oxa-2,6-norbornanecarbolactyl)oxy)-2-oxoethoxy)-2-oxoethyl bromide, 2-(2-(8-(4-oxa-tricyclo[4.3.1.1^{3,8}]undecan-5-one)oxy)-2-oxoethoxy)-2-oxoethyl bromide, 2-(2-(1-adamantylmethoxy)-2-oxoethoxy)-2-oxoethyl bromide, 2-(2-(2-(1-adamantyl)ethoxy)-2-oxoethoxy)-2-oxoethyl bromide, 2-(2-(2-adamantyloxy)-2-oxoethoxy)-2-oxoethyl bromide, 2-(2-(2-methyl-2-adamantyloxy)-2-oxoethoxy)-2-oxoethyl bromide, 2-(2-(2-ethyl-2-adamantyloxy)-2-oxoethoxy)-2-oxoethyl bromide, 2-(2-(2-isopropyl-2-adamantyloxy)-2-oxoethoxy)-2-oxoethyl bromide, 2-(2-(3-hydroxy-1-adamantyloxy)-2-oxoethoxy)-2-oxoethyl bromide, 2-(2-(3,5-dihydroxy-1-adamantyloxy)-2-oxoethoxy)-2-oxoethyl bromide, 2-(2-(3-hydroxy-methyl-1-adamantylmethoxy)-2-oxoethoxy)-2-oxoethyl bromide, 2-(2-(3-carboxyl-1-adamantylmethoxy)-2-oxoethoxy)-2-oxoethyl bromide, 2-(2-(2-cyanomethyl-2-adamantyloxy)-2-oxoethoxy)-2-oxoethyl bromide, 2-(2-(4-oxo-2-adamantyloxy)-2-oxoethoxy)-2-oxoethyl bromide, 2-(2-(5-oxo-2-adamantyloxy)-2-oxoethoxy)-2-oxoethyl bromide, 2-(2-(1-adamantyl)dimethylmethoxy-2-oxoethoxy)-2-oxoethyl bromide, 2-(2-(perfluoropentyloxy)-2-oxoethoxy)-2-oxoethyl bromide, 2-(2-(perfluorocyclohexylloxy)-2-oxoethoxy)-2-oxoethyl bromide, 2-(2-(perfluoro-1-adamantyloxy)-2-oxoethoxy)-2-oxoethyl bromide, 2-(2-(3-hydroxy-perfluoro-1-adamantyloxy)-2-oxoethoxy)-2-oxoethyl bromide, 2-(2-(perfluoro-1-adamantylmethoxy)-2-oxoethoxy)-2-oxoethyl bromide, 2-(2-(3-hydroxy-perfluoro-1-adamantylmethoxy)-2-oxoethoxy)-2-oxoethyl bromide, 2-(1-methyl-2-(2-methyl-2-adamantyloxy)-2-oxoethoxy)-1-methyl-2-oxoethyl bromide, 2-(1-methyl-2-(2-ethyl-2-adamantyloxy)-2-oxoethoxy)-1-methyl-2-oxo ethyl bromide, 2-(1-methyl-2-(2-isopropyl-2-adamantyloxy)-2-oxoethoxy)-1-methyl-2-oxoethyl bromide, 2-(2-(2-methyl-2-adamantyloxy)-2-oxoethoxy)-1-oxoethyl bromide, 2-(2-(2-methyl-2-adamantyloxy)-2-oxoethoxy)ethyl bromide, 2-(2-(2-methyl-2-adamantyloxy)-1-oxoethoxy)-2-oxoethyl bromide, 2-(2-(2-methyl-2-adamantyloxy)-1-oxoethoxy)-1-oxoethyl bromide, 2-(2-(2-methyl-2-adamantyloxy)-1-oxoethoxy)ethyl bromide, 2-(2-(2-methyl-2-adamantyloxy)ethoxy)-2-oxoethyl bromide, 2-(2-(2-methyl-2-adamantyloxy)ethoxy)-1-oxoethyl bromide, 2-(2-(2-methyl-2-adamantyloxy)ethoxy)ethyl bromide, 2-(2-(perfluoro-1-adamantyloxy)ethoxy)-2-oxoethyl bromide, 2-(2-(3-hydroxy-perfluoro-1-adamantyloxy)ethoxy)-2-oxoethyl bromide, 2-(2-(1-adamantyl(dimethylmethoxyethoxy)-2-oxoethyl bromide, 2-(2-(5-(2,6-norbornanecarbolactyl)oxy)ethoxy)-2-oxoethyl bromide, 2-(2-(5-(7-oxa-2,6-norbornanecarbolactyl)oxy)ethoxy)-2-oxoethyl bromide, 2-(2-(cyclopentyloxy)-2-oxoethoxy)-2-oxoethyl chloride, 2-(2-(cyclohexyloxy)-2-oxoethoxy)-2-oxoethyl chloride, 2-(2-(cycloheptyloxy)-2-oxoethoxy)-2-oxoethyl chloride, 2-(2-(cyclooctyloxy)-2-oxoethoxy-2-oxoethyl chloride, 2-(2-(cyclononyloxy)-2-oxoethoxy)-2-oxoethyl chloride, 2-(2-(cyclodecanyloxy)-2-oxoethoxy)-2-oxoethyl chloride, 2-(2-(cyclodecalyloxy)-2-oxoethoxy)-2-oxoethyl chloride, 2-(2-(norbornyloxy)-2-oxoethoxy)-2-oxoethyl chloride, 2-(2-(borntyloxy)-2-oxoethoxy)-2-oxoethyl chloride, 2-(2-(isobornyloxy)-2-oxoethoxy)-2-oxoethyl chloride, 2-(2-(1-adamantyloxy)-2-oxoethoxy)-2-oxoethyl chloride, 2-(2-(3-tricyclo[5.2.1.0^{2,6}]decanyloxy)-2-oxoethoxy)-2-oxoethyl chloride, 2-(2-(3-tetracylo[4.4.0.1^{2,5}1^{7,10}]dodecanyloxy)-2-oxoethoxy)-2-oxoethyl chloride, 2-(2-(1-γ-butyrolactyloxy)-2-oxoethoxy)-2-oxoethyl chloride, 2-(2-(5-(2,6-norbornanecarbolactyl)oxy)-2-oxoethoxy)-2-oxoethyl chloride, 2-(2-(5-(7-oxa-2,6-norbornanecarholactyl)oxy)-2-oxoethoxy)-2-oxoethyl chloride, 2-(2-(8-(4-oxa-tricyclo[4.3.1.1^{3,8}]undecan-5-one)oxy)-2-oxoethoxy)-2-oxoethyl chloride,

2-(2-(1-adamantylmethoxy)-2-oxoethoxy)-2-oxoethyl chloride, 2-(2-(2-(1-adamantyl)ethoxy)-2-oxoethoxy)-2-oxoethyl chloride, 2-(2-(2-adamantyloxy)-2-oxoethoxy)-2-oxoethyl chloride, 2-(2-(2-methyl-2-adamantyloxy)-2-oxoethoxy)-2-oxoethyl chloride, 2-(2-(2-ethyl-2-adamantyloxy)-2-oxoethoxy)-2-oxoethyl chloride, 2-(2-(2-isopropyl-2-adamantyloxy)-2-oxoethoxy)-2-oxoethyl chloride, 2-(2-(3-hydroxy-1-adamantyloxy)-2-oxoethoxy)-2-oxoethyl chloride, 2-(2-(3,5-dihydroxy-1-adamantyloxy)-2-oxoethoxy)-2-oxoethyl chloride, 2-(2-(3-hydroxymethyl-1-adamantylmethoxy)-2-oxoethoxy)-2-oxoethyl chloride, 2-(2-(3-carboxyl-1-adamantylmethoxy)-2-oxoethoxy)-2-oxoethyl chloride, 2-(2-(2-cyanomethyl-2-adamantyloxy)-2-oxoethoxy)-2-oxoethyl chloride, 2-(2-(4-oxo-2-adamantyloxy)-2-oxoethoxy)-2-oxoethyl chloride, 2-(2-(5-oxo-2-adamantyloxy)-2-oxoethoxy)-2-oxoethyl chloride, 2-(2-(1-adamantyl)dimethylmethoxy-2-oxoethoxy)-2-oxoethyl chloride, 2-(2-(perfluorocyclopentyloxy)-2-oxoethoxy)-2-oxoethyl chloride, 2-(2-(perfluorocyclohexyloxy)-2-oxoethoxy)-2-oxoethyl chloride, 2-(2-(perfluoro-1-adamantyloxy)-2-oxoethoxy)-2-oxoethyl chloride, 2-(2-(3-hydroxy-perfluoro-1-adamantyloxy)-2-oxoethoxy)-2-oxoethyl chloride, 2-(2-(perfluoro-1-adamantylmethoxy)-2-oxoethoxy)-2-oxoethyl chloride, 2-(2-(3-hydroxy-perfluoro-1-adamantylmethoxy)-2-oxoethoxy)-2-oxoethyl chloride, 2-(1-methyl-2-(2-methyl-2-adamantyloxy)-2-oxoethoxy)-1-methyl-2-oxoethyl chloride, 2-(1-methyl-2-(2-ethyl-2-adamantyloxy)-2-oxoethoxy)-1-methyl-2-oxoethyl chloride, 2-(1-methyl-2-(2-isopropyl-2-adamantyloxy)-2-oxoethoxy)-1-methyl-2-oxoethyl chloride, 2-(2-(2-methyl-2-adamantyloxy)-2-oxoethoxy)-1-oxoethyl chloride, 2-(2-(2-methyl-2-adamantyloxy)-2-oxoethoxy)ethyl chloride, 2-(2-(2-methyl-2-adamantyloxy)-1-oxoethoxy)-2-oxoethyl chloride,

2-(2-(2-methyl-2-adamantyloxy)-1-oxoethoxy)-1-oxoethyl chloride, 2-(2-(2-methyl-2-adamantyloxy)-1-oxoethoxy)ethyl chloride, 2-(2-(2-methyl-2-adamantyloxy)ethoxy)-2-oxoethyl chloride, 2-(2-(2-methyl-2-adamantyloxy)ethoxy)-1-oxoethyl chloride, 2-(2-(2-methyl-2-adamantyloxy)ethoxy)ethyl chloride, 2-(2-(perfluoro-1-adamantyloxy)ethoxy)-2-oxoethyl chloride, 2-(2-(3-hydroxy-perfluoro-1-adamantyloxy)ethoxy)-2-oxoethyl chloride, 2-(2-(1-adamantyl)dimethylmethoxyethoxy)-2-oxoethyl chloride, 2-(2-(5-(2,6-norbornanecarbolactyl)oxy)ethoxy)-2-oxoethyl chloride, 2-(2-(5-(7-oxa-2,6-norbornanecarbolactyl)oxy)ethoxy)-2-oxoethyl chloride, and the like.

Among these alicyclic structure-containing compounds, preferable from a viewpoint of performance, easiness of production, and the like are 2-(2-(2-methyl-2-adamantyloxy)-2-oxoethoxy)-2-oxoethanol, 2-(2-(2-ethyl-2-adamantyloxy)-2-oxoethoxy)-2-oxoethanol, 2-(2-(2-isopropyl-2-adamantyloxy)-2-oxoethoxy)-2-oxoethanol, 2-(1-methyl-2-(2-methyl-2-adamantyloxy)-2-oxoethoxy)-1-methyl-2-oxoethanol, 2-(1-methyl-2-(2-ethyl-2-adamantyloxy)-2-oxoethoxy)-1-methyl-2-oxoethanol, 2-(1-methyl-2-(2-isopropyl-2-adamantyloxy)-2-oxo ethoxy)-1-methyl-2-oxoethanol, 2-(2-(2-methyl-2-adamantyloxy)-2-oxoethoxy)ethanol, and the like.

Hereinafter, specific examples of chemical formulae of the alicyclic structure-containing compounds of the present invention will be shown. However, the present invention is not limited to these examples.

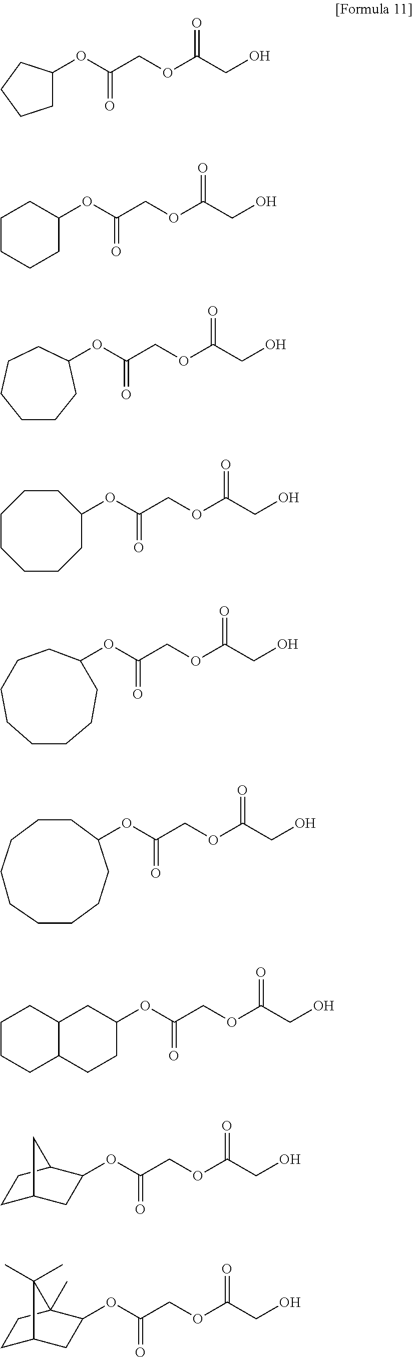

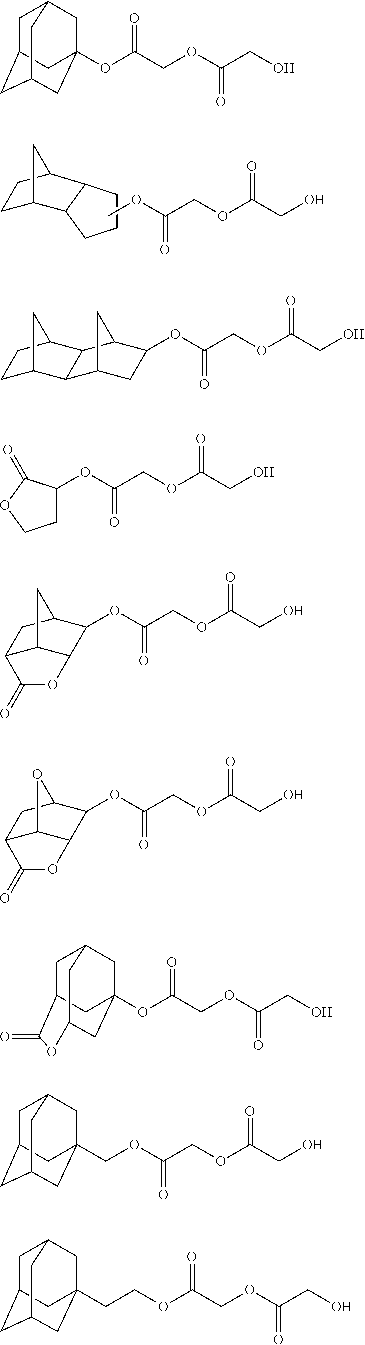

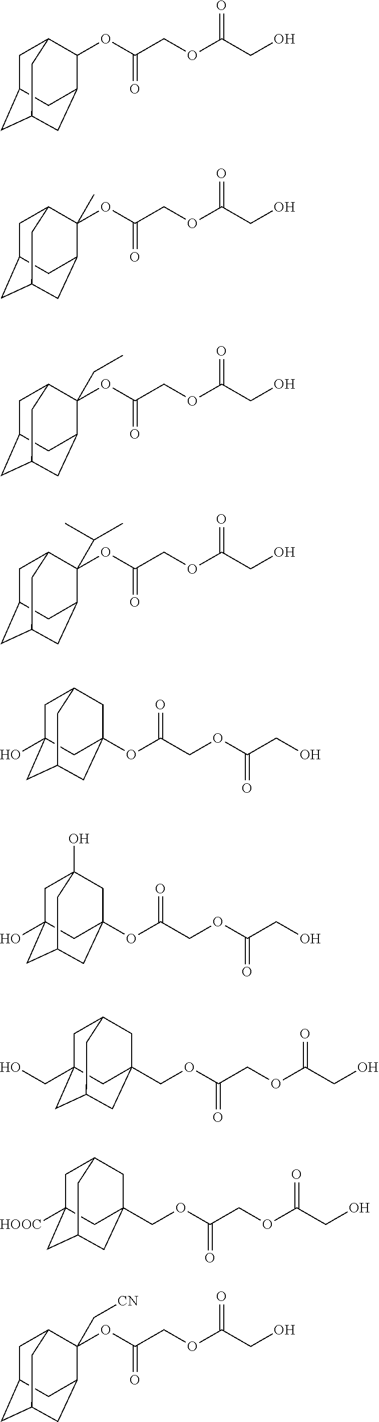

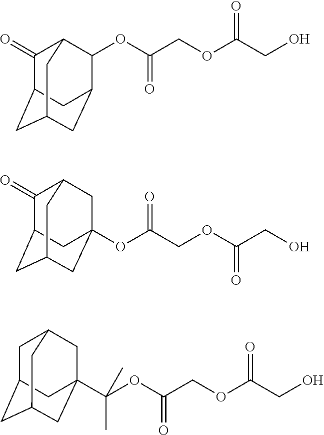 [SMILES: O=C(CO)OCC(=O)OC1C2CC3CC(C2)C(=O)C1C3 ; O=C(CO)OCC(=O)OC12CC3CC(C1)C(=O)C(C3)C2 ; CC(C)(OC(=O)COC(=O)CO)C12CC3CC(CC(C3)C1)C2]

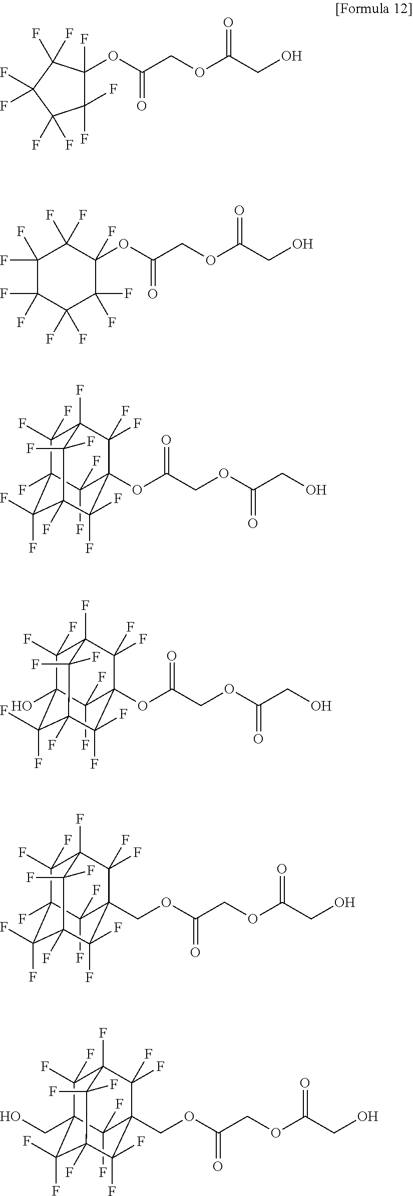 [SMILES: O=C(CO)OCC(=O)OC1(F)C(F)(F)C(F)(F)C(F)(F)C1(F)F ; O=C(CO)OCC(=O)OC1(F)C(F)(F)C(F)(F)C(F)(F)C(F)(F)C1(F)F ; O=C(CO)OCC(=O)OC12C(F)(F)C3(F)C(F)(F)C(F)(C(F)(F)C(F)(C3(F)F)C1(F)F)C2(F)F ; O=C(CO)OCC(=O)OC12C(F)(F)C3(O)C(F)(F)C(F)(C(F)(F)C(F)(C3(F)F)C1(F)F)C2(F)F ; O=C(CO)OCC(=O)OCC12C(F)(F)C3(F)C(F)(F)C(F)(C(F)(F)C(F)(C3(F)F)C1(F)F)C2(F)F ; O=C(CO)OCC(=O)OCC12C(F)(F)C3(F)C(F)(F)C(F)(C(F)(F)C(CO)(C3(F)F)C1(F)F)C2(F)F]

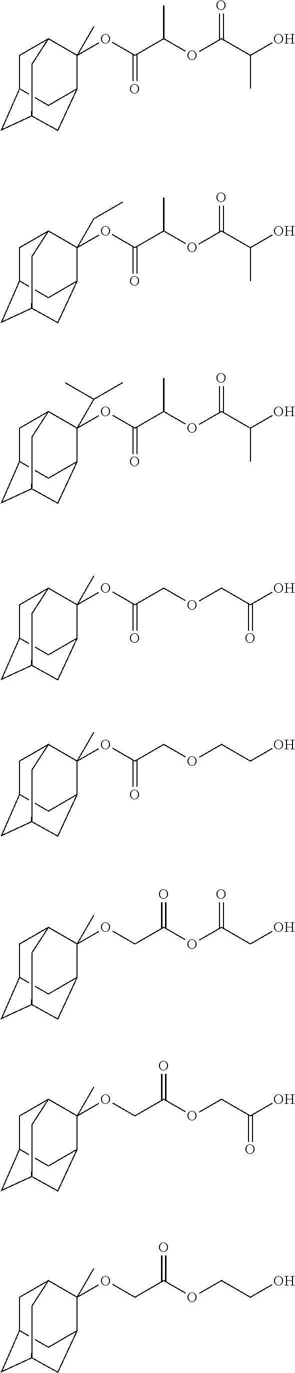

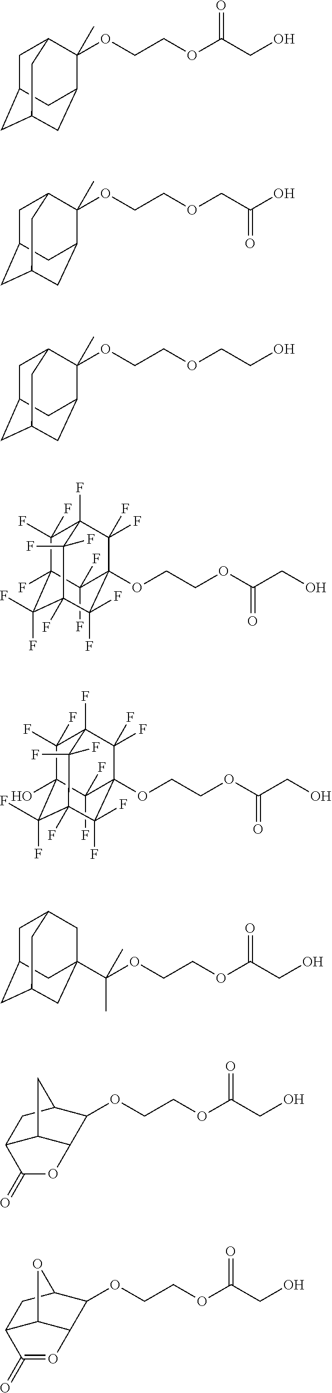

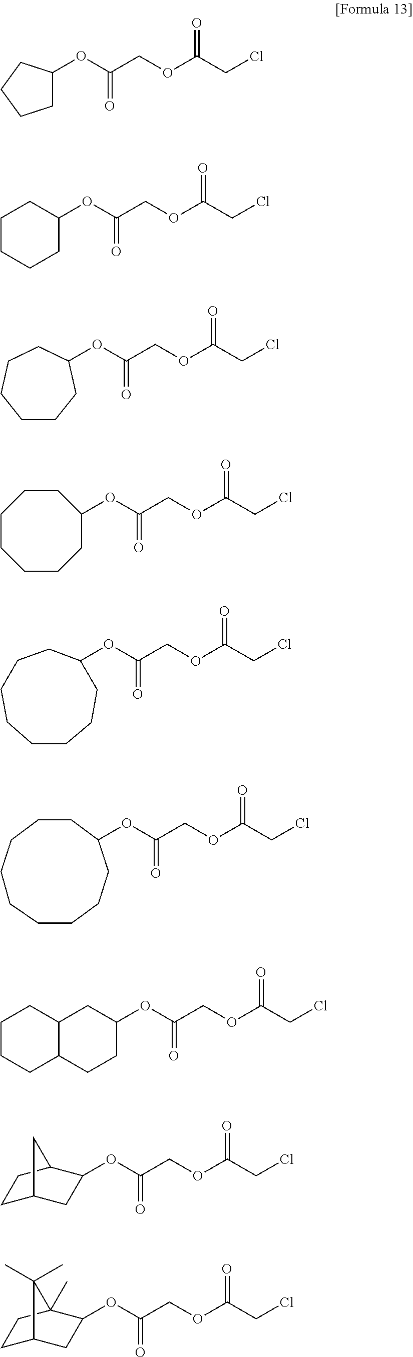

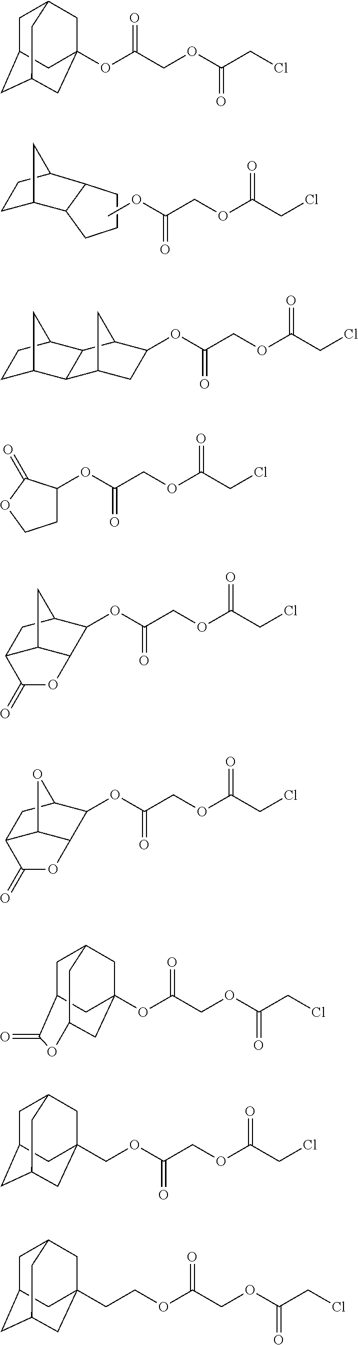

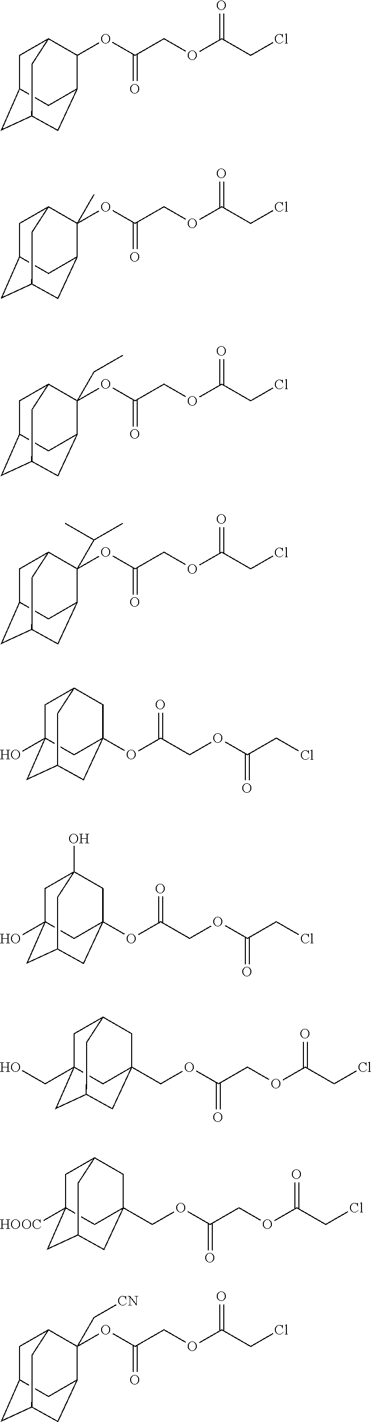

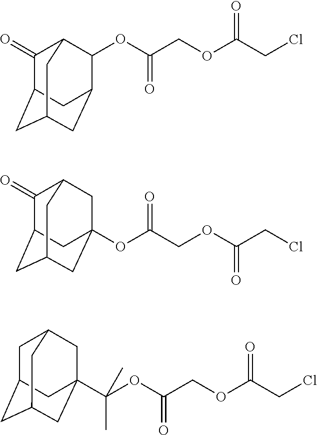 [SMILES: O=C(CCl)OCC(=O)OC1C2CC3CC(C2)C(=O)C1C3 ; O=C(CCl)OCC(=O)OC12CC3CC(C1)C(=O)C(C3)C2 ; CC(C)(OC(=O)COC(=O)CCl)C12CC3CC(CC(C3)C1)C2]

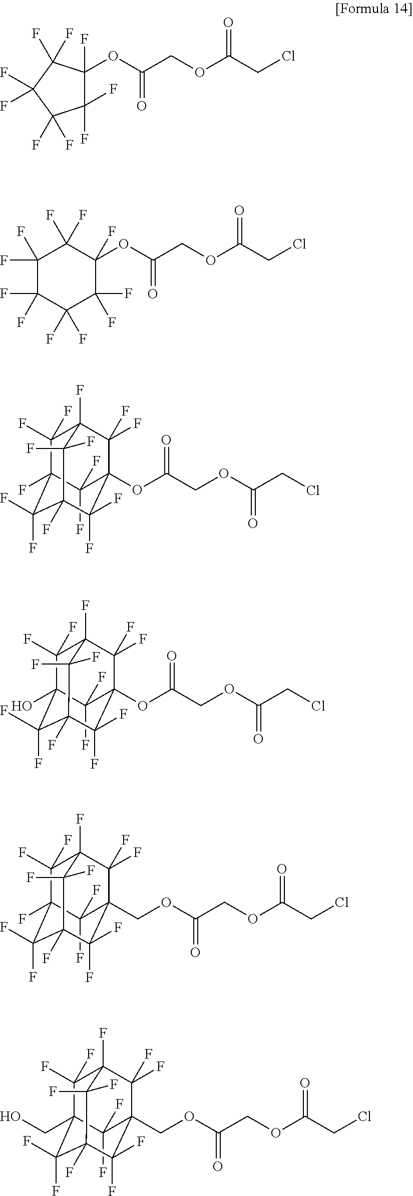 [SMILES: O=C(CCl)OCC(=O)OC1(F)C(F)(F)C(F)(F)C(F)(F)C1(F)F ; O=C(CCl)OCC(=O)OC1(F)C(F)(F)C(F)(F)C(F)(F)C(F)(F)C1(F)F ; O=C(CCl)OCC(=O)OC12C(F)(F)C3(F)C(F)(F)C(F)(C(F)(F)C(F)(C3(F)F)C1(F)F)C2(F)F ; O=C(CCl)OCC(=O)OC12C(F)(F)C3(O)C(F)(F)C(F)(C(F)(F)C(F)(C3(F)F)C1(F)F)C2(F)F ; O=C(CCl)OCC(=O)OCC12C(F)(F)C3(F)C(F)(F)C(F)(C(F)(F)C(F)(C3(F)F)C1(F)F)C2(F)F ; O=C(CCl)OCC(=O)OCC12C(F)(F)C3(F)C(F)(F)C(F)(C(F)(F)C(CO)(C3(F)F)C1(F)F)C2(F)F]

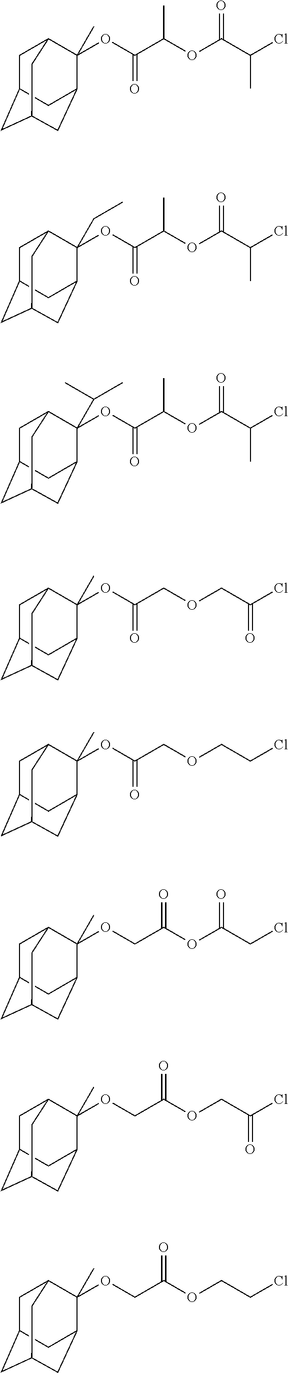

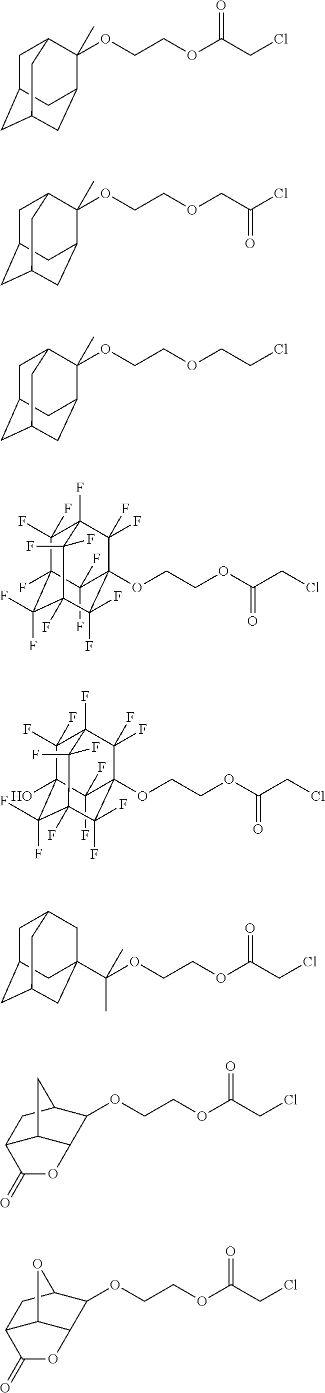

The alicyclic structure-containing compound of the present invention may be produced by various methods. As representative examples, the following methods may be mentioned but the present invention is not limited to these methods.

a. Esterification of an alicyclic structure-containing alcohol with a glycolic acid halide, followed by further esterification with a 2-haloacetic acid halide or a glycolic acid halide.
b. Esterification of an alicyclic structure-containing alcohol with a 2-haloacetic acid halide, followed by further esterification with a 2-haloacetic acid or glycolic acid.
c. Esterification of an alicyclic structure-containing alcohol with a glycolic acid halide, followed by further esterification with a 1,2-dihaloethane or a 2-haloethanol (halohydrin).
d. Esterification of an alicyclic structure-containing alcohol with a 2-haloacetic acid halide, followed by further esterification with a 1,2-dihaloethane or an ethylene glycol.
e. Etherification of an alicyclic structure-containing alcohol with a 1,2-dihaloethane, followed by further esterification with a 2-haloacetic acid halide or glycolic acid.
f. Etherification of an alicyclic structure-containing alcohol with a 1,2-dihaloethane, followed by further etherification with a 2-haloethanol (halohydrin) or an ethylene glycol,
g. Etherification of an alicyclic structure-containing alcohol with a 2-haloethanol (halohydrin), followed by further esterification with a 2-haloacetic acid halide or a glycolic acid halide.
h. Etherification of an alicyclic structure-containing alcohol with a 2-haloethanol (halohydrin), followed by further etherification with a 2-haloethanol (halohydrin) or a 1,2-dilaloethane.
i. Reaction of an alicyclic structure-containing alcohol or an alicyclic structure-containing halogenated hydrocarbon with a dilactide by ring-opening.
j. Esterification of an alicyclic structure-containing alcohol with a 2-haloacetic anhydride.

The above glycolic acid includes aliphatic 2-hydroxy carboxylic acids such as, for example, glycolic acid, lactic acid (2-hydroxypropionic acid), 2-hydroxybutanoic acid, and the like. The 2-haloacetic acid includes 2-halogenated aliphatic carboxylic acids such as, for example, 2-chloroacetic acid, 2-bromoacetic acid, 2-chloropropionic acid, 2-bromopropionic acid, and the like.

The above glycolic acid halide and the above 2-haloacetic acid halide each include carboxylic acid fluoride, carboxylic acid chloride, carboxylic acid bromide, and carboxylic acid iodide of the corresponding carboxylic acid.

The above 1,2-dihaloethane includes symmetrical or unsymmetrical 1,2-halogenated aliphatic hydrocarbons such as, for example, 1,2-dichloroethane, 1,2-dibromoethane, 1,2-diiodo ethane, 1-bromo-2-chloroethane, 1-bromo-2-iodoethane, 1-chloro-2-iodoethane, 1-bromo-2-chloropropne, 1-bromo-2-iodopropane, and the like,

The above 2-haloethanol (halohydrin) includes linear or branched 2-halogenated aliphatic alcohols such as, for example, 2-chloroethanol (chlorohydrin), 2-bromoethanol (bromohydrin), 2-iodoethanol (iodohydrin), 2-chloro-n-propanol, 2-bromo-n-propanol, 2-iodo-n-propanol, 2-chloro-n-butanol, 2-bromo-n-butanol, 2-iodo-n-butanol, 2-chloroisopropanol, 2-bromoisopropanol, 2-iodoisopropanol, 2-chloro-sec-butanol, 2-bromo-sec-butanol, 2-iodo-sec-butanol, 2-chloro-tert-butanol, 2-bromo-tert-butanol, 2-iodo-tert-butanol, and the like.

The above ethylene glycol includes symmetrical or unsymmetrical 1,2-dihydroxy aliphatic hydrocarbons such as, for example, 1,2-ethanediol (ethylene glycol), 1,2-propanediol (propylene glycol), diethylene glycol, and the like.

The above dilactide includes symmetrical or unsymmetrical [1,4]dioxane-2,5-dione compounds such as, for example, [1,4]dioxane-2,5-dione, 3-methyl-[1,4]dioxane-2,5-dione, 3-ethyl-[1,4]dioxane-2,5-dione, 3,6-dimethyl-[1,4]dioxane-2,5-dione, 3,6-diethyl-[1,4]dioxane-2,5-dione, 3-ethyl-6-methyl-[1,4]dioxane-2,5-dione, and the like.

The above 2-haloacetic anhydride includes symmetrical or unsymmetrical 2,2′-dihalogenated aliphatic carboxylic acid anhydrides such as, for example, 2-chloroacetic anhydride, 2-bromoacetic anhydride, 2-iodoacetic anhydride, 2-bromoacetic-2-chloroacetic anhydride, 2-bromoacetic-2-iodoacetic anhydride, 2-chloroacetic-2-iodoacetic anhydride, 2-chloropropionic anhydride, 2-bromopropionic anhydride, 2-iodopropionic anhydride, 2-bromopropionic-2-chloropropionic anhydride, 2-bromopropionic-2-iodopropionic anhydride, 2-chloropropionic-2-iodopropionic anhydride, 2-chloroacetic-2-chloropropionic anhydride, 2-bromoacetic-2-bromopropionic anhydride, 2-iodoacetic-2-iodopropionic anhydride, and the like.

The above esterification and etherification may be carried out by making a base act on an alicyclic structure-containing alcohol and a reagent to generate a salt in the reaction system, But the reaction may also be accelerated by forcibly removing water generated in the reaction out of the system by an azeotropic dehydration reaction.

The above esterification and etherification can be carried out in the presence or absence of an organic solvent. When an organic solvent is used, the concentration of the substrate is preferably adjusted to about 0.1 mol/L to 10 mol/L. The concentration of the substrate of 0.1 mol/L or more is economically preferable because a sufficient amount of product is obtained by using a usual reactor. The concentration of 10 mol/L or less is preferable because it becomes easy to control the temperature of the reaction mixture.

The organic solvent which may be used includes hydrocarbon solvents such as hexane, heptane, cyclohexane, ethylcyclohexane, benzene, toluene, xylene, and the like; ether solvents such as diethyl ether, dibutyl ether, THF (tetrahydrofuran), dioxane, DME (dimethoxyethane), and the like; halogenated solvents such as dichloromethane, carbon tetrachloride, and the like; and aprotic polar solvents such as DMF (N,N-dimethylformamide), DMSO (dimethyl sulfoxide), NMP (N-methyl-2-pyrrolidone), HMPA (hexamethylphosphoric triamide), HMPT (hexamethylphosphorous triamide), carbon disulfide, and the like. These may be used singly or in a combination of two or more kinds.

The above base includes inorganic bases and organic amines such as sodium hydride, sodium hydroxide, potassium hydroxide, sodium carbonate, potassium carbonate, sodium bicarbonate, potassium bicarbonate, silver oxide, sodium phosphate, potassium phosphate, disodium monohydrogen phosphate, dipotassium monohydrogen phosphate, monosodium dihydrogen phosphate, monopotassium dihydrogen phosphate, sodium methoxide, potassium t-butoxide, triethylamine, tributylamine, trioctylamine, pyridine, N,N-dimethylaminopyridine, DBN (1,5-diambicyclo[4.3.0]non-5-ene), DBU (1,8-diazabicyclo[5.4.0]undec-7-ene), and the like.

In the case of an azeotropic dehydration reaction, as the solvent is preferably selected a hydrocarbon solvent such as cyclohexane, ethylcyclohexane, toluene, xylene, and the like. The ratio of the reagent to be charged is about 0.01 to 100 moles, preferably 1 to 1.5 moles per mole of the alicyclic structure-containing alcohol. The amount of the base to be added is about 0.1 to 10 moles, preferably 1 to 1.5 moles per mole of the alicyclic structure-containing alcohol. The reaction temperature is about −200 to about 200° C., preferably −50 to 100° C. In addition, the reaction pressure in terms of absolute pressure is about 0.01 to 10 MPa, preferably, ordinary pressure to 1 MPa. When the reaction time is long, the residence time becomes long and, when the pressure is too high, a special pressure-tight apparatus becomes necessary, with both cases being un-economical.

After the reaction, the reaction mixture is separated into an aqueous and organic layers, and, if necessary, the product is extracted from the aqueous layer. By distilling off the solvent from the reaction mixture under reduced pressure, there is be obtained an alicyclic structure-containing compound of the present invention. This may be purified if needed or may be subjected to the next reaction without purification. The purification methods include distillation, extractive washing, crystallization, activated carbon adsorption, silica gel column chromatography, and the like. From these general purification methods, a choice may be made in consideration of production scale and necessary purity. However, a method by extractive washing or crystallization is preferable because these allow handling at relatively low temperature and treatment of a large amount of a sample at one time.

The ring-opening reaction of the above dilactide is preferably carried out in the presence of a transesterification catalyst. Specific examples of the transesterification catalyst include oxides such as calcium oxide, barium oxide, lead oxide, zinc oxide, zirconium oxide, and the like; hydroxides such as potassium hydroxide, sodium hydroxide, lithium hydroxide, calcium hydroxide, thallium hydroxide, tin hydroxide, lead hydroxide, nickel hydroxide, and the like; halides such as lithium chloride, calcium chloride, tin chloride, lead chloride, zirconium chloride, nickel chloride, and the like; carbonates such as potassium carbonate, rubidium carbonate, cesium carbonate, lead carbonate, zinc carbonate, nickel carbonate, and the like; bicarbonates such as potassium bicarbonate, rubidium bicarbonate, cesium bicarbonate, and the like; phosphate such as sodium phosphate, potassium phosphate, rubidium phosphate, lead phosphate, zinc phosphate, nickel phosphate, and the like; nitrates such as lithium nitrate, calcium nitrate, lead nitrate, zinc nitrate, nickel nitrate, and the like; carboxylates such as lithium acetate, calcium acetate, lead acetate, zinc acetate, nickel acetate, and the like; alkoxides such as sodium methoxide, sodium ethoxide, potassium methoxide, potassium ethoxide, potassium tert-butoxide, calcium methoxide, calcium ethoxide, barium methoxide, barium ethoxide, tetraethoxytitanium, tetrabutoxytitanium, tetra(2-ethylhexoxy)titanium, and the like; acetylacetonate complexes such as lithium acetylacetonate, zirconium acetylacetonate, zinc acetylacetonate, dibutoxytin acetylacetonate, dibutoxytitanium acetylacetonate, and the like; quaternary ammonium alkoxides such as tetramethylammonium methoxide, tetramethylammonium tert-butoxide, trimethylbenzylammonium ethoxide, and the like; dialkyltin compounds such as dimethyltin oxide, methylbutyltin oxide, dibutyltin oxide, dioctyltin oxide, and the like; distannoxanes such as bis(dibutyltin acetate) oxide, bis(dibutyltin laurate) oxide; dialkyltin carboxylates such as dibutyltin diacetate, dibutyltin dilaurate, and the like. These may be used singly or in a combination of two or more kinds.

The (meth)acrylate of the present invention is represented by the following general formula (II):

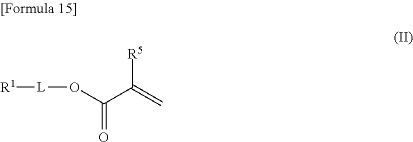

wherein R² represents an alicyclic structure-containing group having 5 to 20 carbon atoms, represented by the following general formula (I); R⁵ represents a hydrogen atom, a methyl group, a fluorine atom, or a trifluoromethyl group; L represents a linking group represented by the following general formula (ii);

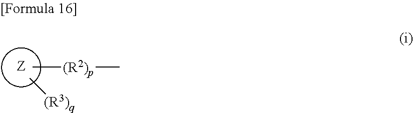

wherein Z represents an alicyclic structure having 5 to 20 carbon atoms, preferably 7 to 12 carbon atoms optionally containing a heteroatom, more preferably an adamantyl ring; R² represents a substituted or unsubstituted bivalent hydrocarbon group having 1 to 5 carbon atoms optionally containing a heteroatom, preferably a bivalent hydrocarbon group having 1 to 2 carbon atoms; R³ represents a substituted or unsubstituted alkyl group optionally containing a heteroatom, a halogen atom, a hydroxyl group, a cyano group, a carboxyl group, an oxo group, or an amino group, preferably a halogen atom, a hydroxyl group, or an oxo group; p represents an integer equal to or larger than 0, preferably 0 to 5, more preferably 0 to 2; q represents an integer equal to or larger than 0, preferably 0 to 20, more preferably 0 to 15; plural R²'s may be the same or different; and plural R³'s may be the same or different;

-{(L^{a})ₗ,(L^{b})ₘ,(L_{c})ₙ}- (ii)

wherein L^{a} represents a linking group represented by the following formula (a); L^{b} represents a linking group represented by the following formula (b); L^{c} represents a linking group represented by the following formula (c); L^{a}, L^{b}, and L^{c} may be bound in any order; and l, m, and n are each independently an integer equal to or larger than 0 and satisfy l+m+n≧2;

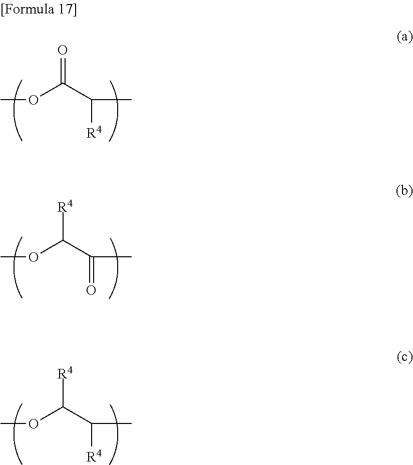

wherein R⁴'s each independently represents a hydrogen atom or a methyl group.

The halogen atoms in the above formula (II) includes a fluorine atom, a chlorine atom, a bromine atom, and an iodine atom.

The alicyclic structure in the above formula (i) having 5 to 20 carbon atoms optionally containing a heteroatom, includes polycyclic lactones such as, for example, a cyclopentyl ring, a cycloheptyl ring, a cycloheptyl ring, a cyclooctyl ring, a cyclononyl ring, a cyclodecanyl ring, a decalyl ring (perhydronaphthalene ring), a norbornyl ring, a bornyl ring, an isobornyl ring, an adamantyl ring, a tricyclo[5.2.1.0^{2,6}]decane ring, tetracyclo[4.4.0.1^{2,5}.1^{7,10}]dodecane ring; 4-oxa-tricyclo[4.2.1.0^{3,7}]nonan-5-one, 4,8-dioxo-tricyclo[4.2.1.0^{3,7}]nonan-5-one, and 4-oxa-tricyclo[4.3.1.1^{3,8}]undecan-5-one; and perfluoro derivative of these; and the like. Among these, preferable is the adamantyl ring.

Specific examples of the substituted or unsubstituted bivalent hydrocarbon group in the above formula (i) having 1 to 5 carbon atoms optionally containing a heteroatom, include linear or branched alkylene groups such as a methylene group, an ethylene group, a trimethylene group; and perfluoro derivatives of these groups.

Specific examples of the substituted or unsubstituted alkyl group in the above formula (i) optionally containing a heteroatom, include linear or branched alkyl groups such as a methyl group, an ethyl group, a n-propyl group, an isopropyl group, a n-butyl group, a sec-butyl group, a tert-butyl group, a n-pentyl group, an isopentyl group, a hexyl group, a heptyl group, an octyl group, a nonyl group, a decanyl group, and the like; and perfluoro derivatives of these.

Specific examples of the above heteroatom, which may be contained by the alicyclic structure having 5 to 20 carbon atoms optionally containing a heteroatom, the substituted or unsubstituted bivalent hydrocarbon group optionally containing a heteroatom, or the substituted or unsubstituted alkyl group optionally containing a heteroatom, include a nitrogen atom, a sulfur atom, an oxygen atom, and the like.

L in the above general formula (II) represents a bivalent linking group represented by the above general formula (ii) and contain the above linking groups L^{a}, L^{b}, and L^{c}. These linking groups may be bound in any order to constitute the linking group L. When the linking group L contains at least a plurality of any one of L^{a}, L^{b}, and L^{c}, the respective linking groups L^{a}'s, L^{b}'s, and L^{c}'s may be the same with or different from each other. In addition, the linking groups of the same kind need not be bound next to each other and, specifically, the binding order may be like L^{a}-L^{b}-L^{a}.

In the above general formula (ii), l, m, and n satisfy l+m+n≧2, preferably l+m+n=2, more preferably 1+n=2 and m=0.

The above L is most preferably a linking group represented by the following general formula (iii):

-L^{a}-L^{a}- (iii)

wherein L^{a} is a linking group represented by the above formula (a).

Furthermore, the (meth)acrylate of the present invention is preferably one represented by any one of the following general formulae (10) to (18):

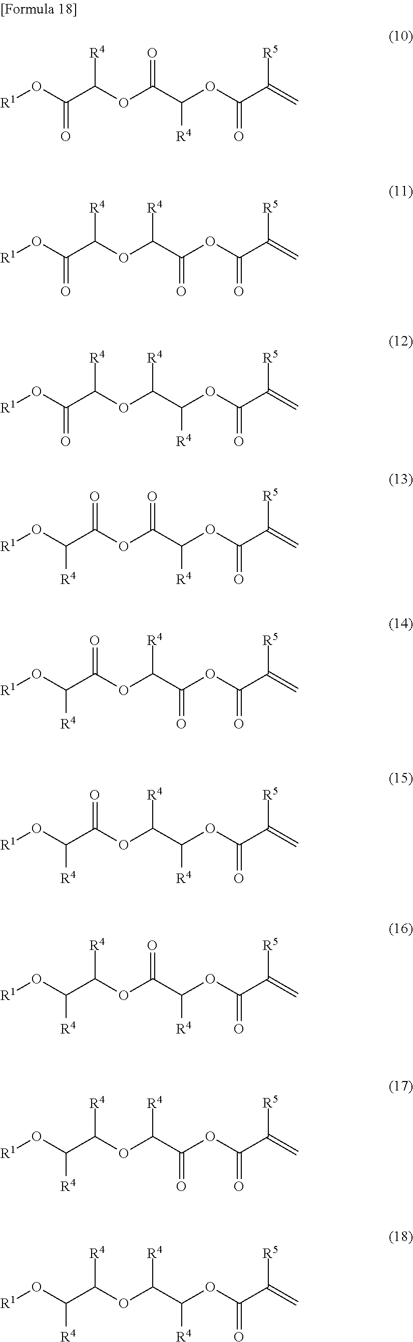

wherein R^{I} represents an alicyclic structure-containing group having 5 to 20 carbon atoms, represented by the above general formula (i); R⁴'s each independently represent a hydrogen atom or a methyl group; and R⁵ represents a hydrogen atom, a methyl group, a fluorine atom, or a trifluoromethyl group.

Specific examples of the (meth)acrylate of the present invention, represented by the above general formulae (10) to (18) include 2-(2-(cyclopentyloxy)-2-oxoethoxy)-2-oxoethyl methacrylate, 2-(2-(cyclohexyloxy)-2-oxoethoxy)-2-oxoethyl methacrylate, 2-(2-(cycloheptyloxy)-2-oxoethoxy)-2-oxoethyl methacrylate, 2-(2-(cyclooctyloxy)-2-oxoethoxy)-2-oxoethyl methacrylate, 2-(2-(cyclononyloxy)-2-oxoethoxy)-2-oxoethyl methacrylate, 2-(2-(cyclodecanyloxy)-2-oxoethoxy)-2-oxoethyl methacrylate, 2-(2-(cyclodecalyloxy)-2-oxoethoxy)-2-oxoethyl methacrylate, 2-(2-(norbornyloxy)-2-oxoethoxy)-2-oxoethyl methacrylate, 2-(2-(bornyloxy)-2-oxoethoxy)-2-oxoethyl methacrylate, 2-(2-(isobornyloxy)-2-oxoethoxy)-2-oxoethyl methacrylate, 2-(2-(1-adamantyloxy)-2-oxoethoxy)-2-oxoethyl methacrylate, 2-(2-(3-tricyclo[5.2.1.0^{2,6}]decanyloxy)-2-oxoethoxy)-2-oxoethyl methacrylate,

2-(2-(3-tetracyclo[4.4.0.1^{2,5}.1^{7,10}]dodecanyloxy)-2-oxoethoxy)-2-oxoethyl methacrylate, 2-(2-(1-γ-butyrolactyloxy)-2-oxoethoxy)-2-oxoethyl methacrylate, 2-(2-(5-(2,6-norbornanecarbolactyl)oxy)-2-oxoethoxy)-2-oxoethyl methacrylate, 2-(2-(5-(7-oxa-2,6-norbornanecarbolactyl)oxy)-2-oxoethoxy)-2-oxoethyl methacrylate, 2-(2-(8-(4-oxa-tricyclo[4.3.1.1^{3,8}]undecan-5-one)oxy)-2-oxoethyl)-2-oxoethyl methacrylate, 2-(2-(1-adamantylmethoxy)-2-oxoethoxy)-2-oxoethyl methacrylate, 2-(2-(2-(1-adamantylethoxy)-2-oxoethoxy)-2-oxoethyl methacrylate, 2-(2-(2-adamantyloxy)-2-oxoethoxy)-2-oxoethyl methacrylate, 2-(2-(2-methyl-2-adamantyloxy)-2-oxoethoxy)-2-oxoethyl methacrylate, 2-(2-(2-ethyl-2-adamantyloxy)-2-oxoethoxy)-2-oxoethyl methacrylate, 2-(2-(2-isopropyl-2-adamantyloxy)-2-oxoethoxy)-2-oxoethyl methacrylate, 2-(2-(3-hydroxy-1-adamantyloxy)-2-oxoethoxy)-2-oxoethyl methacrylate, 2-(2-(3,5-dihydroxy-1-adamantyloxy)-2-oxoethoxy)-2-oxoethyl methacrylate, 2-(2-(3-hydroxymethyl-1-adamantylmethoxy)-2-oxoethoxy)-2-oxoethyl methacrylate, 2-(2-(3-carboxyl-1-adamantylmethoxy)-2-oxoethoxy)-2-oxoethyl methacrylate, 2-(2-(2-cyanomethyl-2-adamantyloxy)-2-oxoethoxy)-2-oxoethyl methacrylate, 2-(2-(4-oxo-2-adamantyloxy)-2-oxoethoxy)-2-oxoethyl methacrylate, 2-(2-(5-oxo-2-adamantyloxy)-2-oxoethoxy)-2-oxoethyl methacrylate, 2-(2-(1-adamantyl)dimethylmethoxy-2-oxoethoxy)-2-oxoethyl methacrylate, 2-(2-(perfluorocyclopentyloxy)-2-oxoethoxy)-2-oxoethyl methacrylate, 2-(2-(perfluorocyclohexyloxy)-2-oxoethoxy)-2-oxoethyl methacrylate,

2-(2-(perfluoro-1-adamantyloxy)-2-oxoethoxy)-2-oxoethyl methacrylate, 2-(2-(3-hydroxy-perfluoro-1-adamantyloxy)-2-oxoethoxy)-2-oxoethyl methacrylate, 2-(2-(perfluoro-1-adamantylmethoxy)-2-oxoethoxy)-2-oxoethyl methacrylate, 2-(2-(3-hydroxy-perfluoro-1-adamantylmethoxy)-2-oxoethoxy)-2-oxoethyl methacrylate, 2-(2-(2-methyl-2-adamantyloxy)-2-oxoethoxy)-2-oxoethyl acrylate, 2-(2-(2-methyl-2-adamantyloxy)-2-oxoethoxy)-2-oxoethyl 2-fluoroacrylate, 2-(2-(2-methyl-2-adamantyloxy)-2-oxoethoxy)-2-oxoethyl 2-trifluoromethylacrylate, 2-(1-methyl-2-(2-methyl-2-adamantyloxy)-2-oxoethoxy)-1-methyl-2-oxoethyl methacrylate, 2-(1-methyl-2-(2-ethyl-2-adamantyloxy)-2-oxoethoxy)-1-methyl-2-oxoethyl methacrylate, 2-(1-methyl-2-(2-isopropyl-2-adamantyloxy)-2-oxoethoxy)-1-methyl-2-oxoethyl methacrylate, 2-(2-(2-methyl-2-adamantyloxy)-2-oxoethoxy)-1-oxoethyl methacrylate, 2-(2-(2-methyl-2-adamantyloxy)-2-oxoethoxy)ethyl methacrylate, 2-(2-(2-methyl-2-adamantyloxy)-1-oxoethoxy)-2-oxoethyl methacrylate,

2-(2-(2-methyl-2-adamantyloxy)-1-oxoethoxy)-1-oxoethyl methacrylate, 2-(2-(2-methyl-2-adamantyloxy)-1-oxoethoxy)ethyl methacrylate, 2-(2-(2-methyl-2-adamantyloxy)ethoxy)-2-oxoethyl methacrylate, 2-(2-(2-methyl-2-adamantyloxy)ethoxy)-1-oxoethyl methacrylate, 2-(2-(2-methyl-2-adamantyloxy)ethoxy)ethyl methacrylate, 2-(2-(perfluoro-1-adamantyloxy)ethoxy)-2-oxoethyl methacrylate, 2-(2-(3-hydroxy-perfluoro-1-adamantyloxy)ethoxy)-2-oxo ethyl methacrylate, 2-(2-(1-adamantyl)dimethylmethoxyethoxy)-2-oxoethyl methacrylate, 2-(2-(5-(2,6-norbornanecarbolactyl)oxy)ethoxy)-2-oxoethyl methacrylate, 2-(2-(5-(7-oxa-2,6-norbornanecarbolactyl(oxy)ethoxy)-2-oxoethyl methacrylate, and the like.

Among these (meth)acrylates, preferable from a standpoint of performance, easiness of production, and the like are 2-(2-(2-methyl-2-adamantyloxy)-2-oxoethoxy)-2-oxoethyl methacrylate, 2-(2-(2-ethyl-2-adamantyloxy)-2-oxoethoxy)-2-oxoethyl methacrylate, 2-(2-(2-isopropyl-2-adamantyloxy)-2-oxoethoxy)-2-oxoethyl methacrylate, 2-(1-methyl-2-(2-methyl-2-adamantyloxy)-2-oxoethoxy)-1-methyl-2-oxoethyl methacrylate, 2-(1-methyl-2-(2-ethyl-2-adamantyloxy)-2-oxoethoxy)-1-methyl-2-oxoethyl methacrylate, 2-(1-methyl-2-(2-isopropyl-2-adamantyloxy)-2-oxoethoxy)-1-methyl-2-oxoethyl methacrylate, 2-(2-(2-methyl-2-adamantyloxy)-2-oxoethoxy)ethyl methacrylate, and the like.

Hereinafter, specific examples of chemical formulae of the (meth)acrylate of the present invention will be shown. However, the present invention is not limited to these examples.

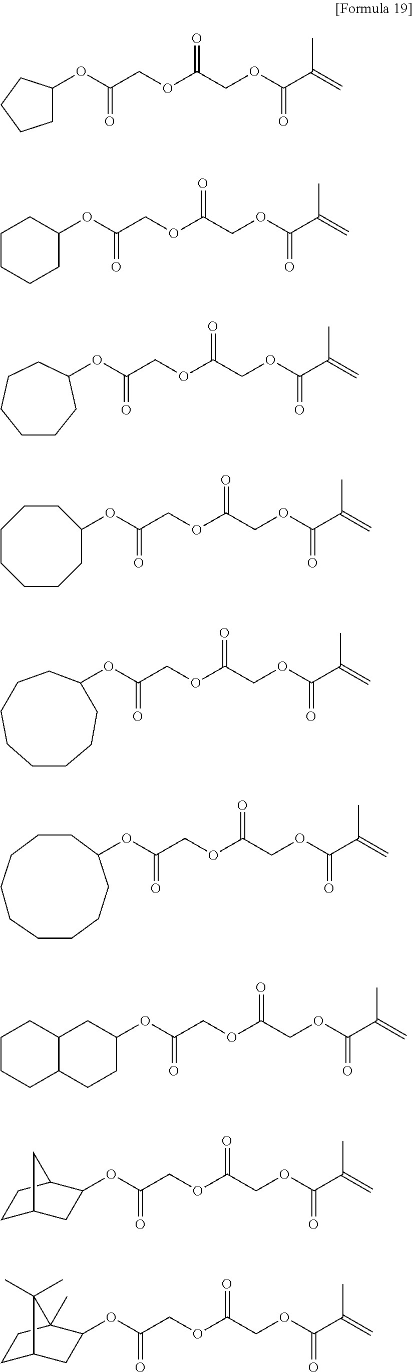

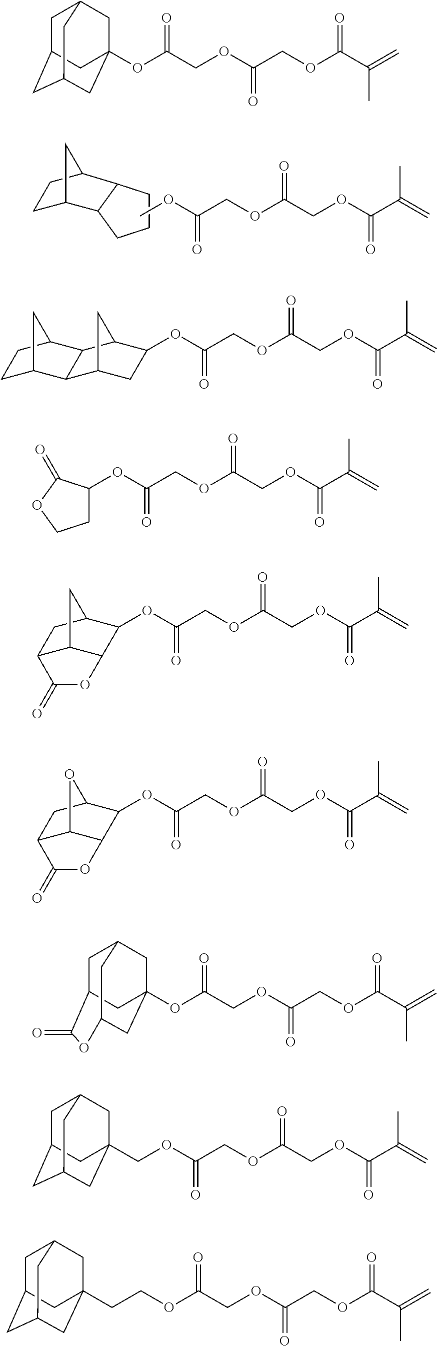

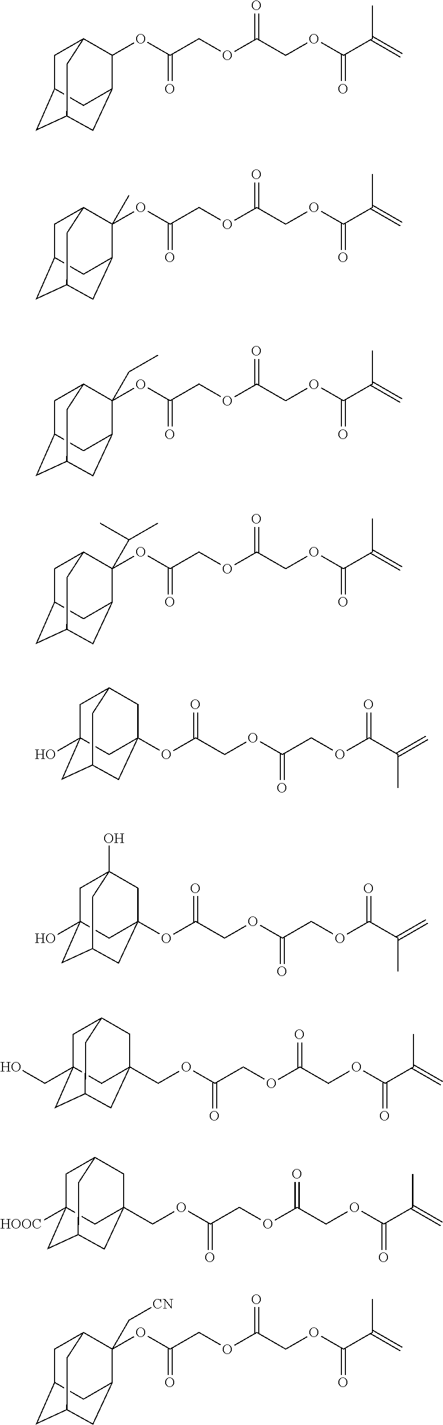

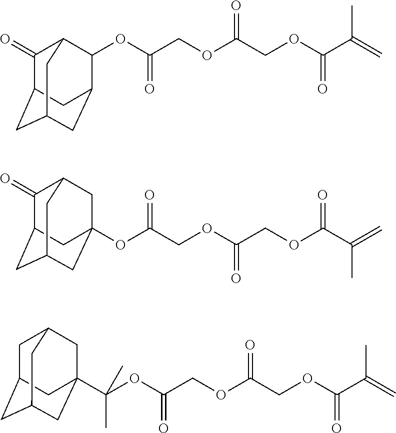 [SMILES: C=C(C)C(=O)OCC(=O)OCC(=O)OC1C2CC3CC(C2)C(=O)C1C3 ; C=C(C)C(=O)OCC(=O)OCC(=O)OC12CC3CC(C1)C(=O)C(C3)C2 ; C=C(C)C(=O)OCC(=O)OCC(=O)OC(C)(C)C12CC3CC(CC(C3)C1)C2]

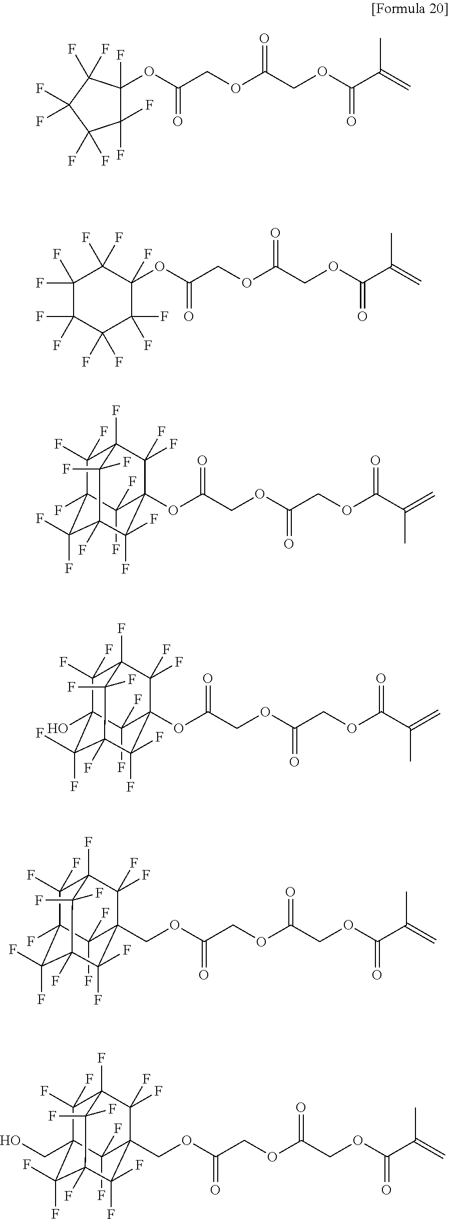 [SMILES: C=C(C)C(=O)OCC(=O)OCC(=O)OC1(F)C(F)(F)C(F)(F)C(F)(F)C1(F)F ; C=C(C)C(=O)OCC(=O)OCC(=O)OC1(F)C(F)(F)C(F)(F)C(F)(F)C(F)(F)C1(F)F ; C=C(C)C(=O)OCC(=O)OCC(=O)OC12C(F)(F)C3(F)C(F)(F)C(F)(C(F)(F)C(F)(C3(F)F)C1(F)F)C2(F)F ; C=C(C)C(=O)OCC(=O)OCC(=O)OC12C(F)(F)C3(O)C(F)(F)C(F)(C(F)(F)C(F)(C3(F)F)C1(F)F)C2(F)F ; C=C(C)C(=O)OCC(=O)OCC(=O)OCC12C(F)(F)C3(F)C(F)(F)C(F)(C(F)(F)C(F)(C3(F)F)C1(F)F)C2(F)F ; C=C(C)C(=O)OCC(=O)OCC(=O)OCC12C(F)(F)C3(F)C(F)(F)C(F)(C(F)(F)C(CO)(C3(F)F)C1(F)F)C2(F)F]

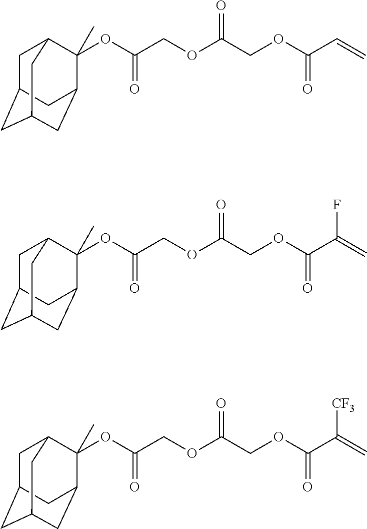 [SMILES: C=CC(=O)OCC(=O)OCC(=O)OC1(C)C2CC3CC(C2)CC1C3 ; C=C(F)C(=O)OCC(=O)OCC(=O)OC1(C)C2CC3CC(C2)CC1C3 ; C=C(C(=O)OCC(=O)OCC(=O)OC1(C)C2CC3CC(C2)CC1C3)C(F)(F)F]

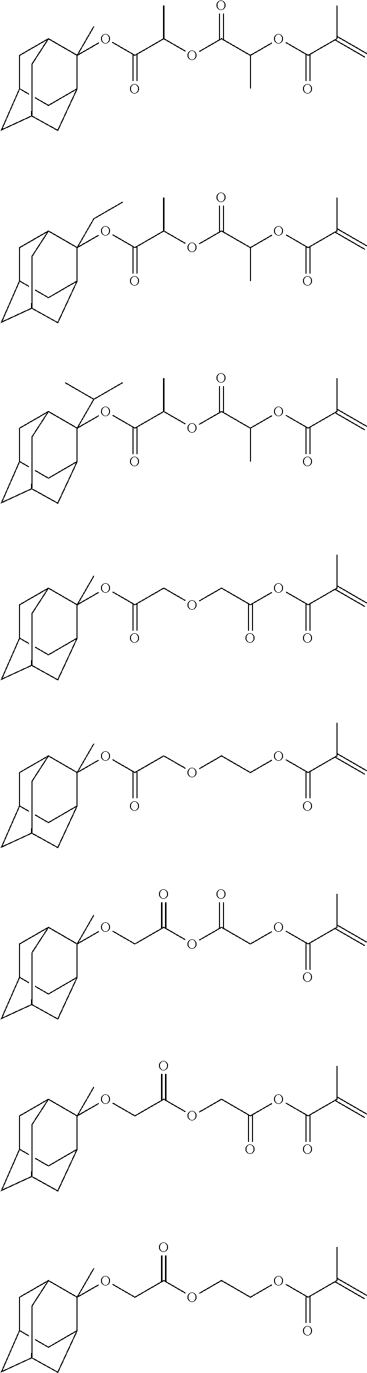

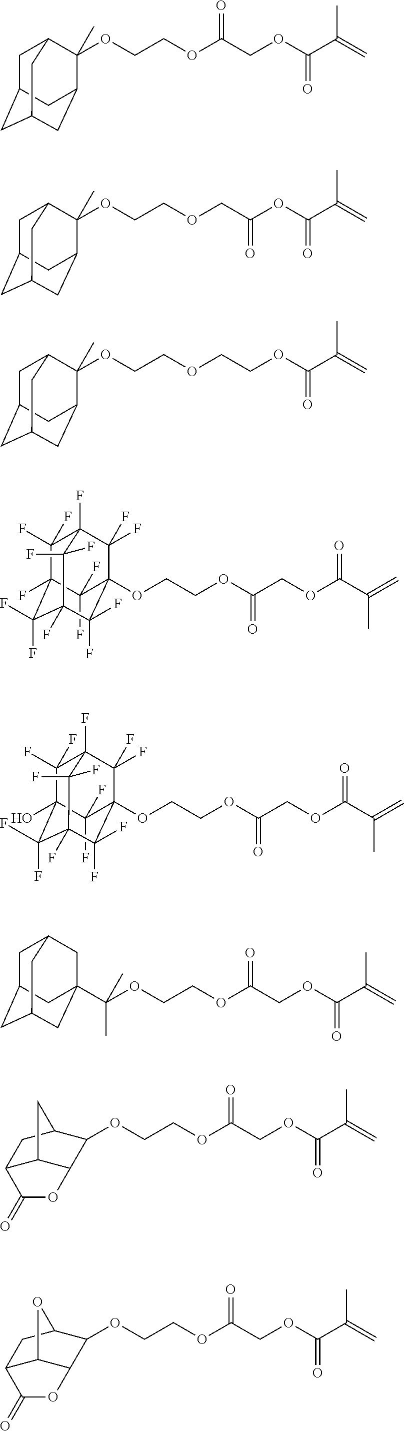

The (meth)acrylate of the present invention may be produced by various methods. Specific examples include the following methods but the present invention is not limited to these methods.

a. Esterification of the alicyclic structure-containing compound of the present invention and one or more selected from (meth)acrylic acid, a (meth)acrylic halide, and a (meth)acrylic anhydride.
b. A transesterification reaction of the alicyclic structure-containing (meth)acrylic acid with a dilactide by a ring opening reaction.

Esterification in production of the (meth)acrylate of the present invention may be carried out by the same method as in esterification in the above production of the alicyclic structure-containing compound of the present invention. Furthermore, the transesterification reaction in production of the (meth)acrylate of the present invention can be carried out by the same method as in the reaction of an alicyclic structure-containing alcohol or an alicyclic structure-containing halogenated hydrocarbon with a dilactide by ring-opening, which is carried out in the above-mentioned production of the alicyclic structure-containing compound of the present invention in the presence of a transesterification catalyst. The reaction temperature is not particularly limited but is preferably 0 to 50° C. When the reaction temperature is 0° C. or higher, the rate of reaction is accelerated and productivity is improved. When the reaction temperature is 50° C. or lower, polymerization of a (meth)acrylic acid can be suppressed. In addition, in order to prevent polymerization of a (meth)acrylic acid throughout from the start of reaction to isolation of the target material, it is preferable to use a radical polymerization inhibitor and, if needed, to bubble air into the reaction mixture.

The alicyclic structure-containing (meth)acrylic acid used in the above transesterification reaction is not particularly limited as long as it is a (meth)acrylic acid having an alicyclic structure. However, one represented by the following general formula (III) is preferable:

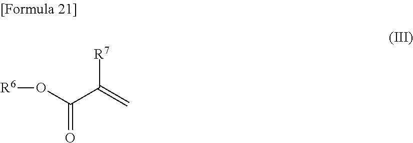

wherein R⁶ represents an alicyclic structure-containing group having 5 to 20 carbon atoms, represented by the following general formula (i); and R⁷ represents a hydrogen atom, a methyl group, a fluorine atom, or a trifluoromethyl group;

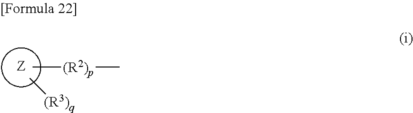

wherein Z represents an alicyclic structure having 5 to 20 carbon atoms optionally containing a heteroatom; R² represents a substituted or unsubstituted bivalent hydrocarbon group having 1 to 5 carbon atoms optionally containing a heteroatom; R³ represents a substituted or unsubstituted alkyl group optionally containing a heteroatom, a halogen atom, a hydroxyl group, a cyano group, a carboxyl group, an oxo group, or an amino group; p and q each independently represent an integer equal to or larger than 0; plural R²'s may be the same or different; and plural R³'s may be the same or different

Specific examples of a dilactide used in the above transesterification reaction include the same ones as the dilactides in the above-described production of alicyclic structure-containing compound of the present invention. [1,4]Dioxane-2,5-dione, 3,6-dimethyl-[1,4]dioxane-2,5-dione, and the like are preferably used.

As the radical polymerization inhibitor mentioned above, there may be used a generally known inhibitor. Specifically, there may be mentioned quinones such as hydroquinone, methoxyhydroquinone, benzoquinone, p-tert-butylcatechol, and the like; alkylphenols such as 2,6-di-tert-butylphenol, 2,4-di-tert-butylphenol, 2-tert-butyl-4,6-dimethylphenol, 2,6-di-tert-butyl-4-methylphenol, 2,4,6-tri-tert-butylphenol, and the like; amines such as alkylated diphenylamine, N,N′-diphenyl-p-phenylenediamine, phenothiazine, 4-hydroxy-2,2,6,6-tetramethylpiperidine, 4-benzoyloxy-2,2,6,6-tetramethylpiperidine, 1,4-dihydroxy-2,2,6,6-tetramethylpiperidine, 1-hydroxy-4-benzoyloxy-2,2,6,6-tetramethylpiperidine, and the like; copper dithiocarbamates such as copper dimethyldithiocarbamate, copper diethyldithiocarbamate, copper dibutyldithiocarbamate, and the like; N-oxyls such as 2,2,6,6-tetramethylpiperidine-N-oxyl, 4-hydroxy-2,2,6,6-tetramethylpiperidine-N-oxyl, 4-benzo yloxy-2,2,6,6-tetramethylpiperidine-N-oxyl, 4-hydroxy-2,2,6,6-tetramethylpiperidine-N-oxyl, and the like.

The method for producing the (meth)acrylate of the present invention includes the method mentioned above as the specific example a or b for producing the (meth)acrylate of the present invention. Namely, the method for producing the (meth)acrylate of the present invention is a method whereby the above alicyclic structure-containing compound and one or more selected from (meth)acrylic acid, a (meth)acrylic acid halide, and a (meth)acrylic anhydride are esterified or the alicyclic structure-containing (meth)acrylic acid is transesterified with a dilactide by a ring-opening reaction to obtain the above (meth)acrylate.

By using the (meth)acrylate of the present invention, there can be obtained a (meth)acrylate polymer.

The (meth)acrylic polymer may be a polymer containing monomer units based on at least one kind of the above (meth)acrylate of the present invention. The polymer may also be a homopolymer obtained by using one kind of the (meth)acrylate of the present invention, a copolymer obtained by using two or more kinds of the (meth)acrylate of the present invention, or a copolymer obtained by using one or more kinds of the (meth)acrylate of the present invention and other polymerizable monomers.

There is no particular limitation to the polymerization method and thus any common polymerization method may be employed. For example, publicly known polymerization methods such as solution polymerization (boiling point polymerization and below-boiling-point polymerization), emulsion polymerization, suspension polymerization, bulk polymerization, and the like may be employed. After polymerization, the less is the amount of a high-boiling point monomer remaining unreacted in the reaction mixture, the more preferable. Thus, during or after completion of the polymerization, it is preferable, if needed, to carry out an operation to remove the unreacted monomer. Among the above polymerization methods, the polymerization reaction which uses a radical polymerization initiator in a solvent is preferable. There is no particular limitation to the polymerization initiator, but a peroxide polymerization initiator, an azo polymerization initiator, and the like may be used.

The peroxide polymerization initiator includes organic peroxides such as a peroxycarbonate, a ketone peroxide, a peroxy ketal, a hydroperoxide, a dialkyl peroxide, a diacyl peroxide, a peroxyester (lauroyl peroxide, benzoyl peroxide), and the like. Furthermore, the azo polymerization initiator includes azo compounds such as 2,2′-azobisisobutyronitrile, 2,2′-azobis(2-methylbutyronitrile), 2,2′-azobis(2,4-dimethylvaleronitrile), dimethyl 2,2′-azobisisobutyrate, and the like.

The above polymerization initiators may suitably be used singly or in a combination of two or more kinds depending on reaction conditions such as polymerization temperature and the like.

After completion of the polymerization, there may be employed various methods as a method to remove the (meth)acrylate of the present invention or other copolymerization monomers used. However, from a viewpoint of operability and economy, preferable is a method whereby the acrylic polymer is washed using a poor solvent for the acrylic polymer. Among poor solvents for the acrylic polymer, low boiling point solvents arc preferable; typically, there may be mentioned methanol, ethanol, n-hexane, n-heptane, and the like.

To the above (meth)acrylic polymer, there may be added a quencher such as a PAG (photoacid generator), an organic amine, and the like; an alkali-soluble components such as an alkali-soluble resin (for example, a novolac resin, a phenol resin, an imide resin, a carboxyl group-containing resin, and the like); a colorant (for example, a dye and the like); an organic solvent (for example, a hydrocarbon, a halogenated hydrocarbon, an alcohol, an ester, a ketone, an ether, a cellosolve, a carbitol, a glycol ether ester, and a mixture of these solvents) and the like to obtain a resin composition, which is suitable for a photoresist.

The photoacid generator includes common compounds which generate acids efficiently by exposure to light, for example, diazonium salts, iodonium salts (for example, diphenyliodonium hexafluorophosphate and the like), sulfonium salts (for example, triphenylsulfonium hexafluoroantimonate, triphenylsulfonium hexafluorophosphate, triphenylsulfonium methanesulfonate, and the like), sulfonates [for example, 1-phenyl-1-(4-methylphenyl)sulfonyloxy-1-benzoylmethane, 1,2,3-trisulfonyloxymethylbenzene, 1,3-dinitro-2-(4-phenylsulfonyloxymethyl)benzene, 1-phenyl-1-(4-methylphenylsulfonyloxymethyl)-1-hydroxy-1-benzoylmethane, and the like], oxathiazole derivatives, s-triazine derivatives, disulfone derivatives (diphenyldisulfone and the like), imide compounds, oxime sulfonates, diazonaphthoquinones, benzoin tosylate, and the like. These photoacid generators may be used singly or in a combination of two or more kinds.

The amount of the photoacid generator used in the above resin composition may be suitably selected depending on the strength of an acid generated by light exposure, the content of a structural unit based on the above (meth)acrylate in the (meth)acrylic polymer, and the like. However, for example, relative to 100 mass parts of the (meth)acrylic polymer, the photoacid generator is contained in an amount of preferably 0.1 to 30 mass parts, more preferably 1 to 25 mass parts, even more preferably 2 to 20 mass parts.

The above resin composition may be prepared by mixing the (meth)acrylic polymer, the photoacid generator, the organic solvent as necessary, and the like, and if needed by removing foreign matter by a common solid separation means such as a filter and the like, A fine pattern may be formed with high accuracy as follows; the resin composition is coated on a substrate or a board, and dried; thereafter, through a predetermined mask, light is exposed on the coated film (resist film) (or further the film is baked after the light exposure) to form a latent image, which is then developed.

The resin composition obtained as described above may be used for various applications such as, for example, a circuit forming material (resist for semiconductor manufacturing; printed circuit board; and the like), an image forming material (printing plate material; relief image; and the like), and the like. Especially, the resin composition is preferably used as a resin composition for a photoresist, more preferably as a resin composition for a positive-type photoresist.

The substrate or the board includes a silicon wafer, metal, plastic, glass, ceramic, and the like. Coating of the resin composition for a photoresist may be carried out by a common coating means such as a spin coater, a dip coater, a roller coater, and the like. Thickness of the coated film, for example, is preferably 0.1 to 20 μm, more preferably 0.3 to 2 μm.

For exposure, there may be used light rays of various wave-lengths, for example, ultraviolet light, X rays, and the like. For semiconductor resists, there may commonly be used g-rays, i-rays, excimer lasers (for example, XeCl, KrF, KrCl, ArF, ArCl), and the like. The exposure energy is, for example, about 1 to 1000 mJ/cm², preferably about 10 to 500 mJ/cm².

By irradiation of light, an acid is generated from the photoacid generator and, by this acid, the cyclic portion in the structure unit based on the (meth)acrylate of formula (I), contained in the (meth)acrylic polymer, is eliminated promptly to generate a carboxyl group which contributes to solubilization. Therefore, by development with water or an alkaline developer, a predetermined pattern may be formed with high accuracy.

### EXAMPLES

Hereinafter, the present invention will be described in more detail by way of Examples and Comparative Examples but the present invention will not be limited to these in any way,

In addition, methods of measurement of physical properties arc as follow.

### (Method of Measurement)

Nuclear magnetic resonance spectroscopy (NMR); measured by JNM-ECA500 (manufactured by JEOL Ltd.) using chloroform-d as a solvent.
Gas chromatography-Mass spectrometry (GC-MS): measured by using EI (manufactured by Shimadzu Corporation, GCMS-QP2010),

### Example 1

### Synthesis of an Alicyclic Structure-Containing Compound: 2-(2-(2-methyl-2-adamantyloxy)-2-oxoethoxy)ethanol

To a 2 L three-necked flask equipped with a thermometer, a condenser, and a stirrer, there were added 100 g (348.2 mmol) of 2-methyl-2-adamantyl bromoacetate, 1000 mL of dimethylformamide, and 389 mL (6975.3 mmol) of ethylene glycol, and the mixture was stirred under a nitrogen atmosphere until complete dissolution. After dissolution, the flask was immersed in an ice bath and the content was cooled to 5° C., whereupon 16.71 g (417.8 mmol) of sodium hydroxide was added. The reaction mixture was warmed to room temperature again and was stirred for 1 hour. After completion of the reaction, 500 ml of a chilled 5 mass % aqueous solution of sodium chloride was added and the mixture was extracted with 1 L of toluene. The organic layer obtained was further washed three times with 500 mL each of 5 mass % aqueous solution of sodium chloride and was concentrated to obtain 78.06 g (yield, 83.5%; GC purity, 91.2%) of the target substance as a light yellow oil.

Results of the measurements of the obtained compound were as follows:

¹H-NMR: 1.59 (d, J=12.6 Hz, 2H), 1.65 (s, 3H), 1.7˜1.98 (m, 10H), 2.31 (m, 2H), 3.69 (m, 2H), 3.74 (m, 2H), 4.09 (s, 21-1);

¹³C-NMR: 22.92, 26.60, 27.27, 33.09, 34.50, 36.25, 38.06, 61.63, 68.61, 73.56, 88.88, 170.13;

GC-MS: 268 (M⁺, 0.05%), 149 (100%), 119 (9.14%)

### Example 2

### Synthesis of a (Meth)Acrylate: 2-(2-(2-methyl-2-adamantyloxy)-2-oxoethoxy)ethyl methacrylate

To a 3 L three-necked flask equipped with a thermometer, a condenser, and a stirrer, there were added 250.1 g (832.0 mmol) of the above 2-(2-(2-methyl-2-adamantyloxy)-2-oxoethoxy)ethanol, 0.25 g (1000 ppm) of p-methoxyphenol, 2000 mL of toluene, and 173.6 mL (1247.2 mmol) of triethylamine, and the mixture was dissolved. After dissolution, the flask was immersed in an ice bath and the content was cooled to 5° C., whereupon 97.5 mL (997.9 mmol) of methacrylic acid chloride was gradually added and the mixture was stirred for 2 hours. After completion of the reaction, 2000 mL of toluene was added and the mixture was washed with 1000 ml of a 10 mass % aqueous solution of potassium carbonate. The organic layer obtained was further washed twice with 1000 mL each of ion-exchanged water and was concentrated to obtain 123.2 g (yield, 45%; GC purity, 96.3%) of the target substance as a colorless oil.

Results of the measurement of the obtained compound were as follows:

¹H-NMR: 1.58 (d, J=12.5 Hz, 2H), 1.65 (s, 3H), 1.71˜1.89 (m, 8H), 1.95 (s, 3H), 1.99 (m, 2H), 2.31 (m, 2H), 3.83 (t, J=5.0 Hz, 2H), 4.09 (s, 2H), 4.34 (t, J=5.0 Hz), 5.58 (s, 1H), 6.15 (s, 1H);

¹³C-NMR: 18.29, 22.39, 26.65, 27.31, 33.09, 34.49, 36.26, 38.10, 63.84, 68.84, 69.41, 88.34, 125.79, 136.11, 167.25, 168.99;

GC-MS: 336 (M⁺, 0.02%), 207 (0.06%), 149 (100.00%), 119 (7.04%), 69 (27.73%)

### Example 3

### Synthesis of an Alicyclic Structure-Containing Compound: 2-(2-(2-methyl-2-adamantyloxy)-2-oxoethoxy)-2-oxoethanol

To a 2 L three-necked flask equipped with a thermometer, a condenser, and a stirrer, there were added 37.6 g (494 mmol) of glycolic acid, 700 mL of DMF, 86.5 g (626 mmol) of potassium carbonate, and 28.3 g (170 mmol) of potassium iodide, and the mixture was stirred at room temperature for 30 minutes. Thereafter, a solution of 100 g (412 mmol) of 2-methyl-2-adamantyl chloroacetate in 300 mL of dimethylformamide was gradually added. The reaction mixture was warmed to 40° C. and was stirred for 4 hours. After completion of the reaction, 2000 mL of diethyl ether was added, the mixture was filtered, and the obtained solution was washed three times with 500 mL each of distilled water. By carrying out crystallization using a mixed solution of toluene (300 mL) and heptane (200 mL), there was obtained 78 g (yield, 67%; GC purity, 99%) of the target substance as a colorless solid.

Results of the measurements of the obtained compound were as follows:

¹H-NMR: 1.59 (d, 21-1, J=12.5 Hz), 1.64 (s, 3H), 1.71˜1.99 (m, 10H), 2.29 (m, 2H), 2.63 (t, 1H, J=5.2H), 4.29 (d, 2H, J=5.2H), 4.67 (s, 2H);

¹³C-NMR: 22,35, 26.56, 27.26, 32.97, 34.54, 36.29, 38.05, 60.54, 61.50, 89.87, 165.97, 172.81;

GC-MS: 282 (M⁺, 0.02%), 165 (0.09%), 149 (40%), 148 (100%), 133 (22%), 117 (2.57%), 89 (0.40%)

### Example 4

### Synthesis of a (Meth)Acrylate: 2-(2-(2-methyl-2-adamantyloxy)-2-oxoethoxy)-2-oxoethyl methacrylate

To a 2 L three-necked flask equipped with a thermometer, a condenser, and a stirrer, there were added 165 g (584 mmol) of 2-(2-(2-methyl-2-adamantyloxy)-2-oxoethoxy)-2-oxoethanol, 2000 mL of THF, 105 mL (754 mmol) of triethylamine, and 0.165 g (1000 ppm) of p-methoxyphenol, and the mixture was dissolved. After dissolution, 62.7 mL (648 mmol) of methacryloyl chloride was gradually added under ice-bath cooling. The mixture was warmed to room temperature and was stirred for 3 hours. After completion of the reaction, 1000 mL of diethyl ether was added and the organic layer was washed five times with 200 ml each of distilled water. The extract was concentrated to obtain 198 g (yield, 97%; GC purity, 99%) of the target substance as a colorless liquid.

Results of the measurements of the obtained compound were as follows:

¹H-NMR: 1.58 (d, J=12.5 Hz, 214), 1.63 (s, 3H), 1.71˜1.89 (m, 8H), 1.98 (s, 3H), 2.00 (m, 2H), 2.30 (m, 214), 4.62 (s, 2H), 4.80 (s, 2H), 5.66 (m, 1H), 6.23 (m, 1H);

¹³C-NMR: 18.04, 22.15, 26.42, 27.14, 32.82, 34.38, 36.11, 37.92, 60.44, 61.28, 89.42, 126.79, 135.18, 165.61, 166.30, 167.20;

GC-MS: 350 (M+, 1.4%), 206 (0.13%), 149 (47%), 148 (100%), 133 (20%), 69 (37%);

### Example 5

### Synthesis of an Alicyclic Structure-Containing Compound: 2-(1-methyl-2-(2-methyl-2-adamantyloxy)-2-oxoethoxy)-1-methyl-2-oxoethanol

To a 1 L three-necked flask equipped with a thermometer, a condenser, and a stirrer, there were added 10 g (0.06 mol) of 2-methyl-2-adamantanol, 6.7 g (0.046 mol) of 3,6-dimethyl-[1,4]dioxane-2,5-dione, and 100 mL of toluene, and the mixture was stirred under a nitrogen atmosphere until complete dissolution. After dissolution, 0.71 g (0.0046 mol) of tin tetrachloride was added and the mixture was stirred under reflux for 5 hours. After completion of the reaction, the reaction mixture was cooled to room temperature (25° C.) and was extracted with diethyl ether. The extract was washed with water and was concentrated to obtain 17 g (yield, 91%) of the target substance as a viscous liquid.

### Example 6

### Synthesis of a (Meth)Acrylate: 2-(1-methyl-2-(2-methyl-2-adamantyloxy)-2-oxoethoxy)-1-methyl-2-oxoethyl methacrylate

To a 1 L three-necked flask equipped with a thermometer, a condenser, and a stirrer, there were added 17 g (0.054 mol) of the above 2-(1-methyl-2-(2-methyl-2-adamantyloxy)-2-oxoethoxy)-1-methyl-2-oxo ethanol, 8.3 g (0.082 mol) of triethylamine, and 200 mL of THF, and the mixture was stirred under a nitrogen atmosphere until complete dissolution. After dissolution, 12.5 g (0.082 mol) of methacrylic anhydride was added and the mixture was stirred at room temperature for 12 hours, After completion of the reaction, the reaction mixture was extracted with diethyl ether and the extract was washed with water. The extract was concentrated and, after purification by column chromatography, there was obtained 13 g (yield, 74%) of the target substance as a viscous liquid.

Results of the measurements of the obtained compound were as follows:

¹H-NMR: 1.50 (d, J=5.8 Hz, 3H), 1.57 (d, J=12.5 Hz, 2H), 1.59 (d, J=5.8 Hz, 3H), 1.61 (s, 3H), 1,71˜1.89 (m, 8H), 1.98 (s, 3H), 2.00 (m, 2H), 2.30 (m, 2H), 4.50 (m, 1H), 4.62 (s, 2H), 4.80 (s, 2H), 4.98 (m, 1H), 5.66 (m, 1H), 6.23 (m, 1H);

¹³C-NMR: 16.52, 16.55, 18.08, 22.20, 26.48, 27.18, 32.91, 34.21, 36.18, 38.02, 66.81, 69.00, 89.43, 126.80, 135.15, 165,60, 165.21, 167.91;

QC-MS: 376 (M+, 0.7%), 220 (0.3%), 149 (52%), 148 (100%), 133 (20%), 69 (37%)

### Example 7

### Synthesis of a (Meth)Acrylate; 2-(1-methyl-2-(2-methyl-2-adamantyloxy)-2-oxoethoxy)-1-methyl-2-oxoethyl methacrylate

To a 1 L three-necked flask equipped with a thermometer, a condenser, and a stirrer, there were added 10 g (0.06 mol) of 2-adamantyl methacrylate (trade name: ADAMANTATE MM, produced by Idemitsu Kosan Co., Ltd.), 2.88 g (0.02 mol) of 3,6-dimethyl-[1,4]dioxane-2,5-dione, 0.01 g of methoquinone, and 100 mL of dichloromethane, and the mixture was stirred under a nitrogen atmosphere until complete dissolution. After dissolution, 0.05 g (0.2 mmol) of tin tetrachloride was added and the mixture was stirred under reflux for 3 hours. After completion of the reaction, the reaction mixture was cooled to room temperature (25° C.) and extracted with diethyl ether. The extract was washed with water and concentrated, and after purification by column, chromatography, there was obtained 4.2 g (yield, 53%) of the target substance as a viscous liquid.

Results of the measurements of the obtained compound were as follows;

¹H-NMR: 1.50 (d, J=5.8 Hz, 3H), 1.57 (d, J=12.5 Hz, 2H), 1.59 (cl, J=5.8 Hz, 3H), 1,61 (s, 3H), 1.71˜1.89 (m, 8H), 1.98 (s, 3H), 2.00 (m, 2H), 2.30 (m, 2H), 4.50 (tn, 1H), 4.62 (s, 2H), 4.80 (s, 2H), 4.98 (m, 1H), 5.66 (m, 1H), 6.23 (m, 1H);

¹C-NMR: 16.52, 16.55, 18.08, 22.20, 26.48, 27.18, 32.91, 34.21, 36.18, 38.02, 66.81, 69.00, 89.43, 126.80, 135.15, 165.60, 165.21, 167.91;

GC-MS: 376 (M+, 0.7%), 220 (0.3%), 149 (52%), 148 (100%), 133 (20%), 69 (37%)

### Reference Example 1

### Synthesis of a (Meth)Acrylic Polymer)

There were charged, as a monomer with a leaving group, 58.50 g of Monomer A obtained in Example 4, represented by the following formula A, and, as a monomer without a leaving group, 28.41 g of Monomer E represented by the following formula E, followed by addition of 1 L of methyl isobutyl ketone to obtain a solution. To the solution, dimethyl 2,2′-azobis(isobutyrate) (V-601) was added in an amount of 1.7 mol % relative to the total amount of monomers and the mixture was heated under reflux (82° C.) for about 2 hours. Subsequently, an operation of pouring the reaction mixture into a large volume of a mixed solvent of methanol and water to precipitate the polymer was repeated three times for purification. As a result, there was obtained a copolymer with copolymer composition (mol) of monomer A:monomer E=43:57, weight average molecular weight (Mw) of 5890, and a polydispersity index (Mw/Mn) of 1.41. The Mw and Mw/Mn are shown in Table 1.

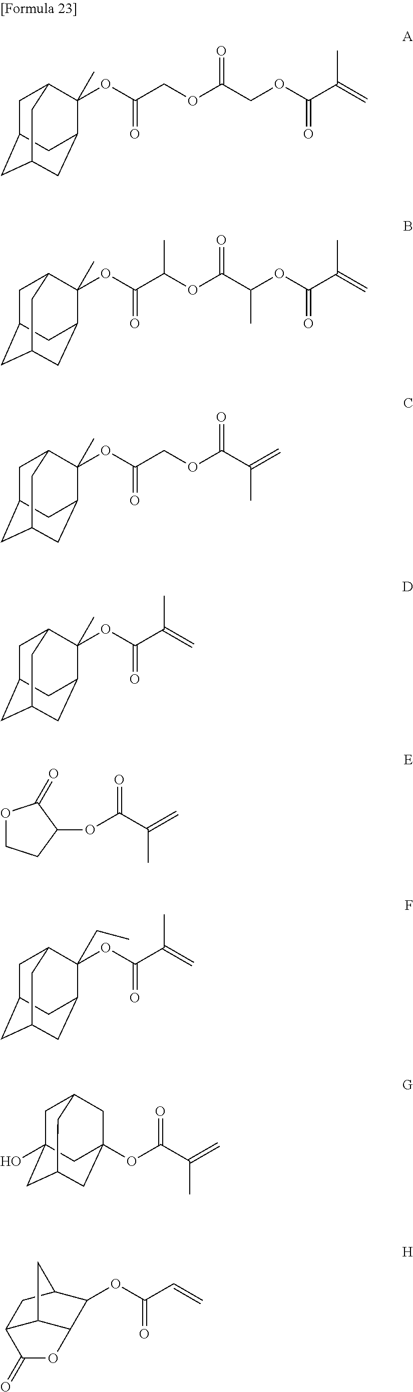

### Reference Example 2

### Synthesis of a (Meth)Acrylic Polymer

In the same manner as in Reference Example 1, using Monomer B obtained in Example 6, represented by the above formula B, there was obtained a copolymer having a monomer composition ratio of a copolymer described in Table 1. The Mw and Mw/Mn are shown in Table 1.

### Comparative Reference Examples 1 and 2

### Synthesis of Polymers

In the same manner as in Reference Example 1, copolymers were obtained with respective monomer composition ratios of copolymers described in Table 1. The Mw's and Mw/Mn's are shown in Table 1.

TABLE 1

| | Monomer | Monomer | | | |
|---|---|---|---|---|---|
| | with a | without a | Copolymer | | |
| | leaving | leaving | composi- | | MW/ |
| | group | group | tion (mol) | Mw | Mn |
| Reference | Monomer A | Monomer E | 43:57 | 5390 | 1.41 |
| Example 1 | | | | | |
| Reference | Monomer B | Monomer E | 47:53 | 6180 | 1.48 |
| Example 2 | | | | | |
| Comparative | Monomer C | Monomer E | 55:45 | 7320 | 1.58 |
| Reference | | | | | |
| Example 1 | | | | | |
| Comparative | Monomer D | Monomer E | 52:48 | 6450 | 1.59 |
| Reference | | | | | |
| Example 2 | | | | | |

### Reference Example 3

### Preparation of a Resin Composition

A resin composition was prepared by mixing 7 g of the (meth)acrylic polymer obtained in Reference Example 1, 0.175 g of triphenylsulfonium nonafluorobutanesulfonate as a photoacid generator, 0.021 g of trioctylamine as a quencher, and 92.8 g of propylene glycol monomethyl ether acetate as a solvent. The prepared resin composition was coated on a silicon wafer and baked at 110° C. for 60 seconds to form a resist film of 250 nm thickness. The thus obtained wafer was subjected to open exposure by light of 248 nm wavelength at several spots at different exposure dose. Immediately after exposure, the wafer was heated at 110° C. for 60 seconds and, thereafter, developed for 60 seconds with an aqueous solution (2.38 mass %) of tetratnethylammonium hydroxide. From the exposed area, a half exposure portion was cut out and its surface roughness (Ra) was measured by using an atomic force microscope (manufactured by Pacific Nanotechnology Inc., Nano-I). The measured value of Ra is shown in Table 2.

### Reference Example 4

### Preparation of a Resin Composition

A resist film was formed in the same manner as in Reference Example 3, except that the (meth)acrylic polymer obtained in Reference Example 2 was used instead of the (meth)acrylic polymer obtained in Reference Example 1. The measurement result of Ra is shown in Table 2.

### Comparative Reference Example 3

### Preparation of a Resin Composition

A resist film was formed in the same manner as in Reference Example 3, except that the (meth)acrylic polymer obtained in Comparative Reference Example 1 was used instead of the (meth)acrylic polymer obtained in Reference Example 1. The measurement result of Ra is shown in Table 2.

### Comparative Reference Example 4

### Preparation of a Resin Composition

A resist film was formed in the same manner as in Reference Example 3, except that the (meth)acrylic polymer obtained in Comparative Reference Example 2 was used instead of the (meth)acrylic polymer obtained in Reference Example 1. The measurement result of Ra is shown in Table 2.

TABLE 2

| | Surface roughness Ra (nm) |
|---|---|
| Reference Example 3 | 0.58 |
| Reference Example 4 | 0.63 |
| Comparative Reference Example 3 | 1.32 |
| Comparative Reference Example 4 | 1.43 |

As described above, the resist prepared by using a polymer which contains the monomer of the present invention shows less surface roughness after development, thus indicating that the monomer of the present invention has a high roughness improvement effect.

### Reference Example 5

### Synthesis of a (Meth)Acrylic Polymer

In a 500 mL beaker, there were charged 18.05 g (106.17 mmol) of Monomer E represented by the aforementioned formula E, 20.06 g (80.89 mmol) of Monomer F represented by the aforementioned formula F, 15.04 g (42.97 mmol) of Monomer A obtained in Example 4 and represented by the aforementioned formula A, and 5.37 g (22.75 mmol) of Monomer G represented by the aforementioned formula G, and the charge was dissolved in 234.08 g of methyl ethyl ketone. To this solution, 17.7 mmol of dimethyl 2,2′-azobis(isobutyrate) (V-601) was added and dissolved. This reaction mixture was added dropwise, under a nitrogen atmosphere over a 6 hour period, to 97.53 g of methyl ethyl ketone which was heated to 75° C. in a separable flask. After the dropwise addition was complete, the reaction mixture was stirred under heating for 1 hour and, thereafter, the reaction mixture was cooled to room temperature. After concentrating the obtained polymerization solution under reduced pressure, an operation was carried out to make the reaction product (a copolymer) separate out by adding the residue dropwise to a large amount of a mixed solution of methanol/water. The reaction product which precipitated was collected by filtration, washed, and dried to obtain 35 g of the desired copolymer.

For this copolymer, the mass average molecular weight (Mw) reduced to standard polystyrene was 8,900 and the polymer distribution (Mw/Mn) was 1.95, as obtained by GPC measurement.

In addition, the copolymer composition ratio (proportion (molar ratio) of each component having the above structural formula) obtained by ¹³C-NMR was Monomer E:Monomer F:Monomer A:Monomer G=52.4:19.6:18.7:9.4.

### Comparative Reference Example 5

### Synthesis of a Polymer

A desired copolymer was obtained in the same manner as in Reference Example 5, except that, in Reference Example 5, Monomer A was not used and Monomer H was used instead of Monomer E.

With this copolymer, the mass average molecular weight (Mw) reduced to standard polystyrene was 10,000 and the polymer distribution (Mw/Mn) was 2.00 as obtained by GPC measurement.

In addition, the copolymer composition ratio (proportion (molar ratio) of each component having the above structural formula) obtained by ¹³C-NMR was Monomer H:Monomer F:Monomer G=40.0:40.0:20.0.

### Reference Example 6

### Preparation of a Resin Composition

A resin composition was prepared by mixing 10 g of the (meth)acrylic polymer obtained in Reference Example 5, 0.467 g of 4-methylphenyldiphenylsulfonium nonafluorobutanesulfonate as a photoacid generator, and 220 g of a mixed solution of propylene glycol monomethyl ether acetate/propylene glycol monomethyl ether=6/4 as a solvent.

On an 8 inch silicon wafer, an organic antireflective coat composition (trade name: “ARC29”, produced by Brewer Science Inc.) was coated by using a spinner and dried by baking on a hot plate at 205° C. for 60 seconds to form an organic antireflective coat of 82 nm thickness. Then, on the antireflective coat, the resin composition obtained above was coated by using a spinner and dried by a prebake (PAB) treatment on a hot plate under conditions of 90° C. and 60 seconds to form a resist film with a film thickness of 120 nm.

Subsequently, an ArF excimer laser (193 nm) was selectively irradiated on the resist film through a mask pattern (6% halftone) by means of an ArF exposure device NSR-S302 (manufactured by Nikon Inc.; NA (numeric aperture)=0.60, 2/3 ring band illumination).

Then, the resist film was subjected to a post exposure baking (PEB) treatment at 90° C. for 60 second, and, further, to an alkaline development under the conditions at 23° C. with a 2.38 mass % aqueous solution of tetramethylammonium hydroxide (TMAH) (product name:NMD-3, produced by Tokyo Ohka Kogyo Co., Ltd.) for 30 seconds, thereafter rinsed with pure water for 30 seconds, and dried by spinning off.

As a result, on the resist film was formed a resist pattern, namely a contact hole pattern having holes of 130 nm diameter disposed at regular intervals (pitch, 260 nm).

In this case, there was obtained an optimum exposure amount, Eop (mJ/cm²; sensitivity), for the formation of a contact hole pattern having a diameter of 130 nm and a pitch of 260 nm. The results are shown in Table 3.

In addition, each contact hole pattern formed above was observed from above by using a scanning electron microscope (SEM) and circularity of the hole pattern was evaluated according to the following criteria. The results are included in Table 3.

Good: the hole pattern as a whole has high circularity and has good shape.

Acceptable; distortion is seen in a part of the hole pattern and has somewhat inferior circularity.

### Comparative Reference Example 6

### Preparation of a Resin Composition

A resist film was formed, the shape of the hole pattern was observed, and optimum exposure amount was obtained in the same manner as in Reference Example 6, except that the (meth)acrylic polymer obtained in Comparative Reference Example 5 was used instead of the (meth)acrylic polymer obtained in Reference Example 5. The results are shown together in Table 3.

TABLE 3

| | Optimum exposure | Hole pattern |
|---|---|---|
| | dose Eop (mJ/cm²) | shape |
| Reference Example 6 | 7 | Good |
| Comparative Reference Example 6 | 30 | Acceptable |

### INDUSTRIAL APPLICABILITY

The alicyclic structure-containing compound and the (meth)acrylate of the present invention are particularly excellent as a resist material which corresponds to short-wavelength irradiation light.

1.-7. (canceled)

8. A method for producing a (meth)acrylate comprising esterifying an alicyclic compound and one or more compounds selected from the group consisting of (meth)acrylic acid, a (meth)acrylic acid halide, and a (meth)acrylic anhydride,
wherein said alicyclic compound is represented by the following formula (I):
R¹-L-X (I)
wherein R¹ represents an alicyclic group having 5 to 20 carbon atoms, represented by the following formula (i); L represents a linking group represented by the following formula (ii); and X represents a halogen atom or a hydroxyl group; 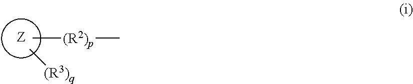
wherein Z represents an alicyclic structure having 5 to 20 carbon atoms optionally comprising a heteroatom; R² represents a substituted or unsubstituted bivalent hydrocarbon group having 1 to 5 carbon atoms optionally comprising a heteroatom; R³ represents a substituted or unsubstituted alkyl group optionally comprising a heteroatom, a halogen atom, a hydroxyl group, a cyan group, a carboxyl group, an oxo group, or an amino group; p and q each independently represent an integer equal to or larger than 0; plural R²'s may be the same or different; and plural R³'s may be the same or different;
-{(L^{a})ₗ,(L^{b})ₘ,(L_{c})ₙ}- (ii)
wherein L^{a} represents a linking group represented by the following formula (a); L^{b} represents a linking group represented by the following formula (b); L^{c} represents a linking group represented by the following formula (c); and L^{a}, L^{b}, and L^{c} may be bound in any order, and l, m, and n represent each independently an integer equal to or larger than 0 and satisfy l+m+n≧2; 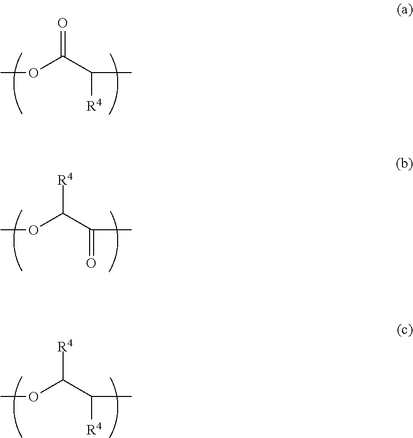
wherein R⁴'s each independently represent a hydrogen atom or a methyl group.

9. A method for producing a (meth)acrylate wherein the (meth)acrylate is obtained by a process comprising transesterifying an alicyclic structure-containing (meth)acrylic acid and dilactide by a ring-opening reaction.

10. The method of claim 8, wherein the alicyclic compound is represented by any one of formulae (1) to (9): 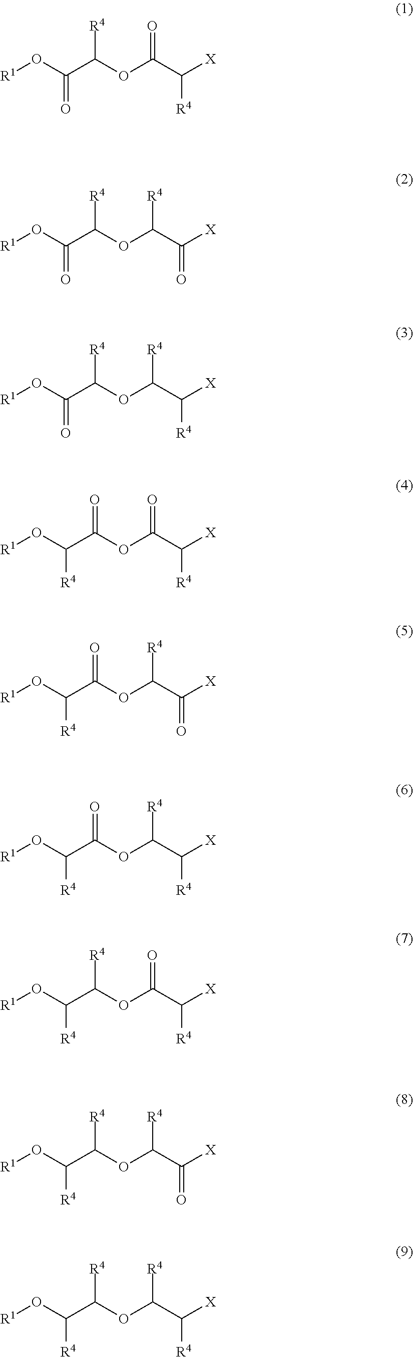
wherein R¹ represents an alicyclic group having 5 to 20 carbon atoms, represented by said formula (i); R⁴'s each independently represent a hydrogen atom or a methyl group; and X represents a halogen atom or a hydroxyl group.

11. The method of claim 9, wherein the alicyclic structure-containing (meth)acrylic acid is represented by the following general formula (III) 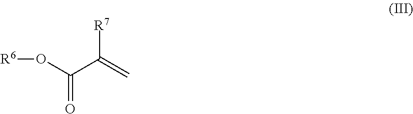
wherein R⁶ represents an alicyclic structure-containing group having 5 to 20 carbon atoms, represented by the following general formula (i); and R⁷ represents a hydrogen atom, a methyl group, a fluorine atom, or a trifluoromethyl group; 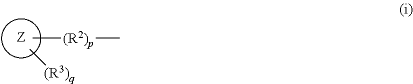
wherein Z represents an alicyclic structure having 5 to 20 carbon atoms optionally containing a heteroatom; R² represents a substituted or unsubstituted bivalent hydrocarbon group having 1 to 5 carbon atoms optionally containing a heteroatom; R³ represents a substituted or unsubstituted alkyl group optionally containing a heteroatom, a halogen atom, a hydroxyl group, a cyano group, a carboxyl group, an oxo group, or an amino group; p and q each independently represent an integer equal to or larger than 0; plural R²'s may be the same or different; and plural R³'s may be the same or different.

12. The method of claim 9, wherein the dilactide is [1,4]Dioxane-2,5-dione or 3,6-dimethyl-[1,4]dioxane-2,5-dione.
